(19) United States
(12) Patent Application Publication
(10) Pub. No.: US 2013/0129720 A1
(43) Pub. Date: May 23, 2013

(54) Combination Cancer Therapies with Wortmannin Analogs

(71) Applicants: Scott Peterson, Woodinville, WA (US); Diana F. Hausman, Seattle, WA (US); Robert Kirkman, Yarrow Point, WA (US)

(72) Inventors: Scott Peterson, Woodinville, WA (US); Diana F. Hausman, Seattle, WA (US); Robert Kirkman, Yarrow Point, WA (US)

(73) Assignee: ONCOTHYREON INC., Seattle, WA (US)

(21) Appl. No.: 13/701,290

(22) PCT Filed: Jun. 3, 2011
(86) PCT No.: PCT/US11/39159

§ 371 Date: Feb. 6, 2013

Related U.S. Application Data
(60) Provisional application No. 61/351,559, filed on Jun. 4, 2010.
(60) Provisional application No. 61/408,995, filed on Nov. 1, 2010.
(60) Provisional application No. 61/416,148, filed on Nov. 22, 2010.
(60) Provisional application No. 61/416,157, filed on Nov. 22, 2010.
(60) Provisional application No. 61/416,037, filed on Nov. 22, 2010.

(51) Int. Cl. A61K 31/366; A61K 39/395; A61K 45/06; A61K 31/337; A61K 31/56

(52) U.S. Cl. CPC A61K 31/366; A61K 31/337; A61K 31/56; A61K 45/06; A61K 39/39558
USPC 424/133.1; 514/453; 514/171

(57) ABSTRACT

Provided herein are combination therapies for the treatment of certain cancers in a subject by administering a combination of a therapeutic and a wortmannin analog to that subject.

### CROSS-REFERENCE

This application claims the benefit of U.S. Provisional Application No. 61/351,559, filed Jun. 4, 2010; U.S. Provisional Application No. 61/416,037, filed Nov. 22, 2010; U.S. Provisional Application No. 61/408,995, filed Nov. 1, 2010, U.S. Provisional Application No. 61/416,148, filed Nov. 22, 2010; and U.S. Provisional Application No. 61/416,157, filed Nov. 22, 2010, each of which is incorporated herein by reference in its entirety.

### BACKGROUND OF THE INVENTION

Phosphatidylinositol-3-kinase (PI-3K) signaling is activated in a broad spectrum of human cancers via multiple mechanisms, including the increased expression or activity of cell surface receptors that activate PI-3K, increased expression of the PI-3K catalytic subunit, as well as mutations that activate the catalytic subunit or suppress the capacity of the regulatory subunit to regulate catalytic subunit activity. In addition, loss of PTEN via mutation, deletion, or epigenetic suppression serves to drive the pathway downstream of PI-3K. In addition to the genetic and histological evidence for PI-3K pathway activation in human cancer samples, PI-3K activation has been shown to be oncogenic in mouse cancer models. Taken together, it is contemplated that PI-3K pathway activation contributes to human disease pathology.

### SUMMARY OF THE INVENTION

Provided herein are methods for treating cancer in a subject with a combination therapy of a wortmannin analog with another therapeutic. Also provided herein, are certain dosing regimens for treatment of cancers with a combination therapy of a wortmannin analog with another therapeutic. In certain embodiments, the therapeutic is a taxoid. In other embodiments, the therapeutic is a epidermal growth factor receptor (EGFR) inhibitor.

Provided herein, in one aspect, are methods for treating cancer in a subject with a combination therapy of a taxoid and a wortmannin analog. Cancers to be treated with a combination therapy described herein, include head and neck cancer, lung cancer, ovarian cancer, liver cancer, colon cancer, breast cancer, pancreatic cancer, kidney cancer, cervical cancer, uterine cancer, prostate cancer, esophageal cancer and gastric cancer. Provided herein also are methods for reducing solid tumors in a subject with cancer with a combination therapy of a taxoid and a wortmannin analog. Also provided herein are methods of improving or maintaining the quality of life in a subject with cancer with a combination therapy of a taxoid and a wortmannin analog.

In one aspect, provided herein are methods for treating subject with a locally advanced, recurrent or metastatic cancer comprising administering to the subject a compound selected from

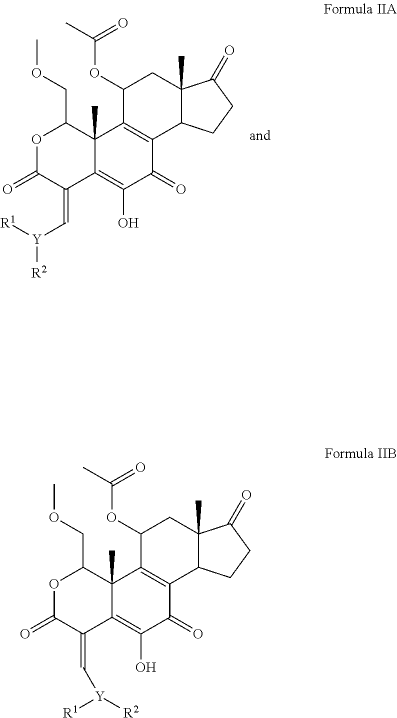

wherein Y is a heteroatom selected from nitrogen and sulfur and R¹ and R² are independently selected from an unsaturated alkyl, cyclic alkyl, or R¹ and R² together with Y form a heterocycle, and docetaxel.

In some embodiments, the cancer is selected from the group consisting of head and neck cancer, lung cancer, ovarian cancer, liver cancer, colon cancer, breast cancer, pancreatic cancer, kidney cancer, cervical cancer, uterine cancer, prostate cancer, esophageal cancer and gastric cancer. In other embodiments, the cancer is unresectable. In certain instances, the cancer is non-small cell lung cancer (NSCLC). In certain instances, the cancer is head and neck squamous cell carcinoma (SCCHN). In further instances, the cancer is incurable.

In another aspect, provided herein are methods for treating subject with a locally advanced, recurrent or metastatic non-small cell lung cancer comprising administering to the subject a compound selected from

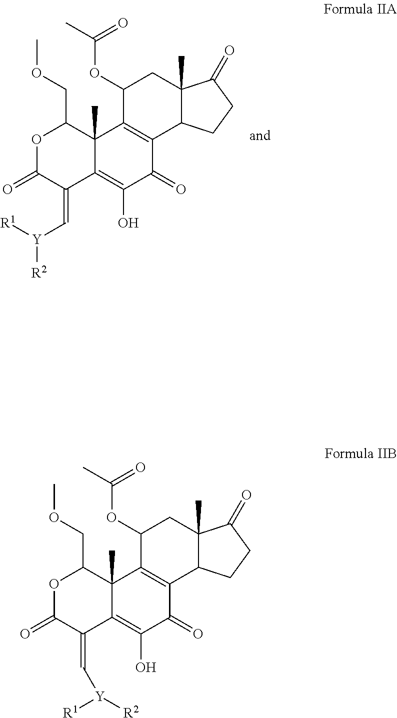

wherein Y is a heteroatom selected from nitrogen and sulfur and R¹ and R² are independently selected from an unsaturated alkyl, cyclic alkyl, or R¹ and R² together with Y form a heterocycle, and docetaxel.

In another aspect, provided herein are methods for treating subject with a locally advanced, recurrent or metastatic head and neck squamous cell carcinoma comprising administering to the subject a compound selected from

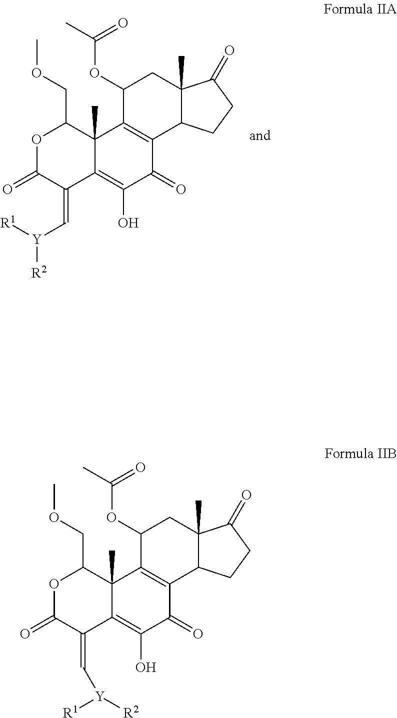

wherein Y is a heteroatom selected from nitrogen and sulfur and R¹ and R² are independently selected from an unsaturated alkyl, cyclic alkyl, or R¹ and R² together with Y form a heterocycle, and docetaxel.

In another aspect, provided herein are methods for treating subject with a locally advanced, recurrent or metastatic colorectal cancer comprising administering to the subject a compound selected from

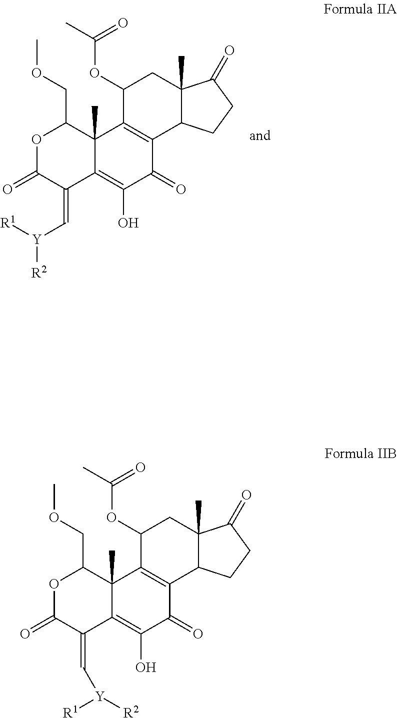

wherein Y is a heteroatom selected from nitrogen and sulfur and R¹ and R² are independently selected from an unsaturated alkyl, cyclic alkyl, or R¹ and R² together with Y form a heterocycle, and docetaxel.

In some embodiments of the methods provided herein, docetaxel and the compound are administered simultaneously. In other embodiments, docetaxel and the compound are administered sequentially. In further embodiments, docetaxel and the compound are administered in a single composition.

In some embodiments of the methods provided herein, administering of the compound is by injection, transdermal, nasal, pulmonary, vaginal, rectal, buccal, ocular, otic, local, topical, or oral delivery. In certain instances, injection is intramuscular, intravenous, subcutaneous, intranodal, intratumoral, intracisternal, intraperitoneal, or intradermal.

In some embodiments of the methods provided herein, docetaxel and the compound are administered via different routes of administration. In certain embodiments, the compound is administered orally. In certain embodiments, the compound is administered in a capsule form. In certain embodiments, the compound administered in about 0.1 to about 12 mg. In certain instances, the compound is administered daily.

In some embodiments of the methods provided herein, docetaxel is administered intravenously. In some embodiments, docetaxel is administered periodically every three weeks. In some embodiments, docetaxel is administered weekly.

In some embodiments, the administration is over a period of time selected from the group consisting of at least about 3 weeks, at least about 6 weeks, at least about 9 weeks, at least about 12 weeks, at least about 15 weeks, at least about 18 weeks, at least about 21 weeks, at least about 24 weeks, at least about 27 weeks, at least about 30 weeks, at least about 33 weeks, at least about 36 weeks, at least about 39 weeks, at least about 42 weeks, at least about 45 weeks, at least about 48 weeks, at least about 51 weeks, at least about 54 weeks, at least about 57 weeks, at least about 60 weeks, at least about 75 weeks, at least about 90 weeks, and at least about 120 weeks.

In further embodiments of the methods provided herein, docetaxel and compound is provided in a kit. In further embodiments, a subject is pretreated with a corticosteroid prior to administration of docetaxel and the compound. In yet further embodiments, the methods provided herein further are an adjuvant to doxorubicin, cyclophosphamide and/or fluorouracil therapy. In yet further embodiments, the methods provided herein further comprise an anti-emetic, anti-diarrheal or both.

In further embodiments of the methods provided herein, the subject is preselected for having completed first-line anti-cancer therapy. In other embodiments, subject is preselected for sensitivity to administration of the compound. In certain instances, preselection is by assessment of genetic mutations in PI-3 kinase, PTEN and/or K-ras genes.

In other embodiments of the methods provided herein, the methods further comprise evaluating the treated subject, wherein the evaluation comprises determining at least one of: (a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life.

In some embodiments of the methods provided herein, the compound is

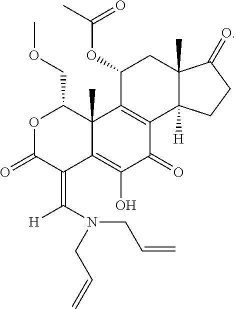 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

In certain instances wherein the compound is

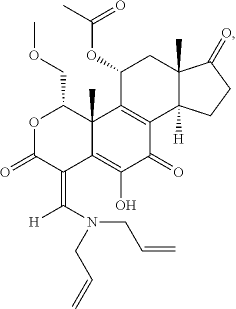 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

the compound is administered at a dose and frequency sufficient to result in one or more of the following: 1) 17-hydroxy metabolite between about 500 pg/mL and about 2500 pg/mL (peak) within about 1-3 hours of administration; 2) plasma Cₘₐₓ of the 17-hydroxy metabolite of between about 750 pg/mL and about 1750 pg/mL; and 3) AUC of between about 2000 hr*pg/mL and about 8000 hr*pg/mL for the 17-hydroxy metabolite.

In other embodiments of the methods provided herein, the compound is

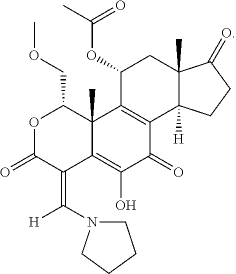 [SMILES: COC[C@H]1OC(=O)/C(=C/N2CCCC2)C2=C(O)C(=O)C3=C([C@H](OC(C)=O)C[C@]4(C)C(=O)CC[C@@H]34)[C@]21C]

In some embodiments, the compound is administered as a continuous dose. In other embodiments, the compound is administered as an intermittent dose. It further embodiments, the compound is administered as a combination of a continuous and intermittent dose.

In another aspect, provided herein are methods for reducing solid tumors in a subject diagnosed with a locally advanced, recurrent or metastatic cancer comprising administering to the subject a compound selected from

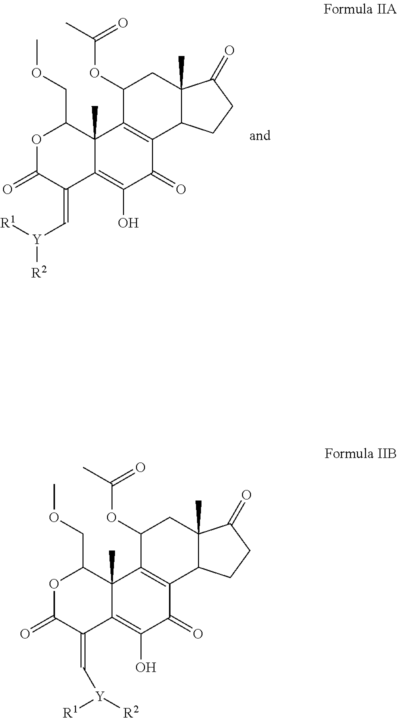

wherein Y is a heteroatom selected from nitrogen and sulfur and R¹ and R² are independently selected from an unsaturated alkyl, cyclic alkyl, or R¹ and R² together with Y form a heterocycle, and docetaxel.

In yet another aspect, provided herein are methods for improving or maintaining the quality of life of a subject diagnosed with a locally advanced, recurrent or metastatic cancer comprising administering to the subject a compound selected from

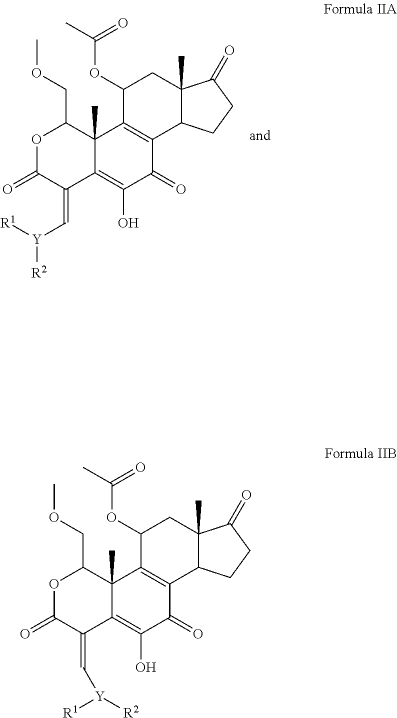

wherein Y is a heteroatom selected from nitrogen and sulfur and R¹ and R² are independently selected from an unsaturated alkyl, cyclic alkyl, or R¹ and R² together with Y form a heterocycle, and docetaxel.

Also provided herein, in another aspect, are methods for treating cancer in a subject with a combination therapy of an epidermal growth factor receptor (EGFR) inhibitor and a wortmannin analog. Cancers to be treated with a combination therapy described herein, include head and neck cancer, lung cancer, ovarian cancer, liver cancer, colon cancer, breast cancer, pancreatic cancer, kidney cancer, cervical cancer, uterine cancer, prostate cancer, esophageal cancer and gastric cancer. Provided herein also are methods for reducing solid tumors in a subject with cancer with a combination therapy of an EGFR inhibitor and a wortmannin analog. Also provided herein are methods of improving or maintaining the quality of life in a subject with cancer with a combination therapy of an EGFR inhibitor and a wortmannin analog.

In one aspect, provided herein are methods for treating subject with a locally advanced, recurrent or metastatic cancer comprising administering to the subject a compound selected from

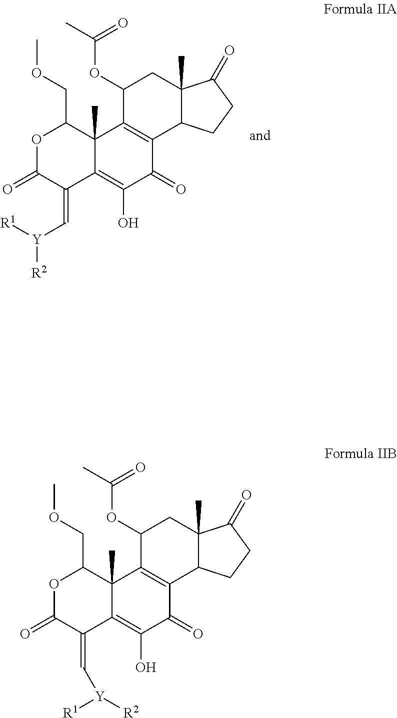

wherein Y is a heteroatom selected from nitrogen and sulfur and R¹ and R² are independently selected from an unsaturated alkyl, cyclic alkyl, or R¹ and R² together with Y form a heterocycle, and cetuximab.

In some embodiments, the cancer is selected from the group consisting of head and neck cancer, lung cancer, ovarian cancer, liver cancer, colon cancer, breast cancer, pancreatic cancer, kidney cancer, cervical cancer, uterine cancer, prostate cancer, esophageal cancer and gastric cancer. In other embodiments, the cancer is unresectable. In certain instances, the cancer is non-small cell lung cancer (NSCLC). In certain instances, the cancer is head and neck squamous cell carcinoma (SCCHN). In certain instances, the cancer is colorectal cancer. In further instances, the cancer is incurable.

In another aspect, provided herein are methods for treating subject with a locally advanced, recurrent or metastatic non-small cell lung cancer comprising administering to the subject a compound selected from

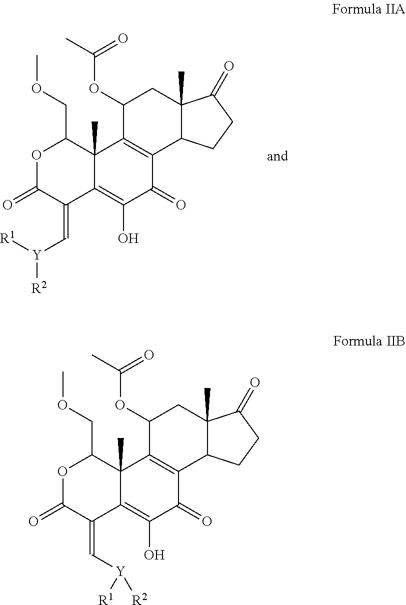

wherein Y is a heteroatom selected from nitrogen and sulfur and R¹ and R² are independently selected from an unsaturated alkyl, cyclic alkyl, or R¹ and R² together with Y form a heterocycle, and cetuximab.

In another aspect, provided herein are methods for treating subject with a locally advanced, recurrent or metastatic head and neck squamous cell carcinoma comprising administering to the subject a compound selected from

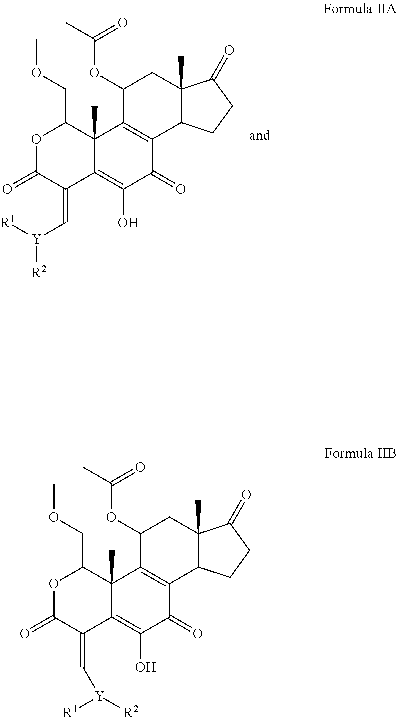

wherein Y is a heteroatom selected from nitrogen and sulfur and R¹ and R² are independently selected from an unsaturated alkyl, cyclic alkyl, or R¹ and R² together with Y form a heterocycle, and cetuximab.

In another aspect, provided herein are methods for treating subject with a locally advanced, recurrent or metastatic colorectal cancer comprising administering to the subject a compound selected from

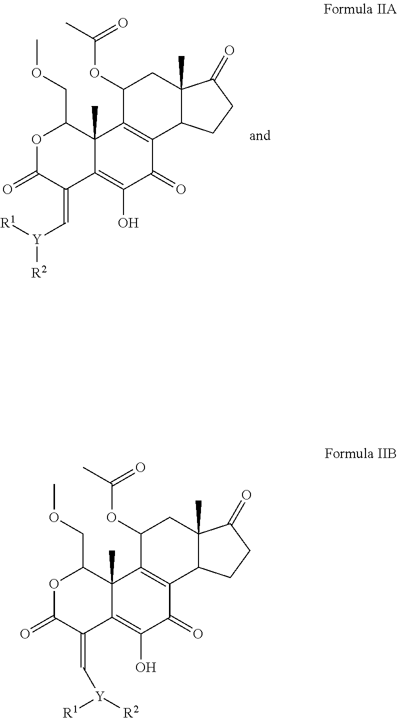

wherein Y is a heteroatom selected from nitrogen and sulfur and R¹ and R² are independently selected from an unsaturated alkyl, cyclic alkyl, or R¹ and R² together with Y form a heterocycle, and cetuximab.

In some embodiments of the methods provided herein, cetuximab and the compound are administered simultaneously. In other embodiments, cetuximab and the compound are administered sequentially. In further embodiments, cetuximab and the compound are administered in a single composition.

In some embodiments of the methods provided herein, administering of the compound is by injection, transdermal, nasal, pulmonary, vaginal, rectal, buccal, ocular, otic, local, topical, or oral delivery. In certain instances, injection is intramuscular, intravenous, subcutaneous, intranodal, intratumoral, intracisternal, intraperitoneal, or intradermal.

In some embodiments of the methods provided herein, cetuximab and the compound are administered via different routes of administration. In certain embodiments, the compound is administered orally. In certain embodiments, the compound is administered in a capsule form. In certain embodiments, the compound administered in about 0.1 to about 12 mg. In certain instances, the compound is administered daily.

In some embodiments of the methods provided herein, cetuximab is administered intravenously. In some embodiments, cetuximab is administered periodically every three weeks. In some embodiments, cetuximab is administered weekly.

In some embodiments, the administration is over a period of time selected from the group consisting of at least about 3 weeks, at least about 6 weeks, at least about 9 weeks, at least about 12 weeks, at least about 15 weeks, at least about 18 weeks, at least about 21 weeks, at least about 24 weeks, at least about 27 weeks, at least about 30 weeks, at least about 33 weeks, at least about 36 weeks, at least about 39 weeks, at least about 42 weeks, at least about 45 weeks, at least about 48 weeks, at least about 51 weeks, at least about 54 weeks, at least about 57 weeks, at least about 60 weeks, at least about 75 weeks, at least about 90 weeks, and at least about 120 weeks.

In further embodiments of the methods provided herein, cetuximab and compound is provided in a kit. In yet further embodiments, the methods provided herein further comprise a topoisomerase inhibitor. In yet further embodiments, the methods provided herein further comprise irinotecan. In yet further embodiments, the methods provided herein further comprise an anti-emetic, anti-diarrheal or both.

In some embodiments of the methods provided herein, the subject is pretreated with an H₁ antagonist. In further embodiments of the methods provided herein, the subject is preselected for having completed first-line anti-cancer therapy. In other embodiments, subject is preselected for sensitivity to administration of the compound. In certain instances, preselection is by assessment of genetic mutations in PI-3 kinase, PTEN and/or K-ras genes.

In other embodiments of the methods provided herein, the methods further comprise evaluating the treated subject, wherein the evaluation comprises determining at least one of: (a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life.

In some embodiments of the methods provided herein, the compound is

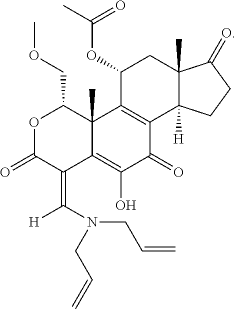 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

In certain instances wherein the compound is

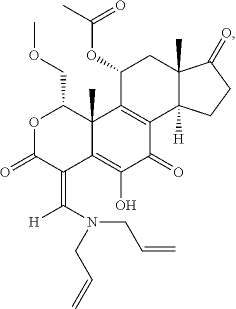 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

the compound is administered at a dose and frequency sufficient to result in one or more of the following: 1) 17-hydroxy metabolite between about 500 pg/mL and about 2500 pg/mL (peak) within about 1-3 hours of administration; 2) plasma Cₘₐₓ of the 17-hydroxy metabolite of between about 750 pg/mL and about 1750 pg/mL; and 3) AUC of between about 2000 hr*pg/mL and about 8000 hr*pg/mL for the 17-hydroxy metabolite.

In other embodiments of the methods provided herein, the compound is

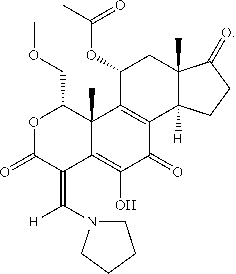 [SMILES: COC[C@H]1OC(=O)/C(=C/N2CCCC2)C2=C(O)C(=O)C3=C([C@H](OC(C)=O)C[C@]4(C)C(=O)CC[C@@H]34)[C@]21C]

In some embodiments, the compound is administered as a continuous dose. In other embodiments, the compound is administered as an intermittent dose. It further embodiments, the compound is administered as a combination of a continuous and intermittent dose.

In another aspect, provided herein are methods for reducing solid tumors in a subject diagnosed with a locally advanced, recurrent or metastatic cancer comprising administering to the subject a compound selected from

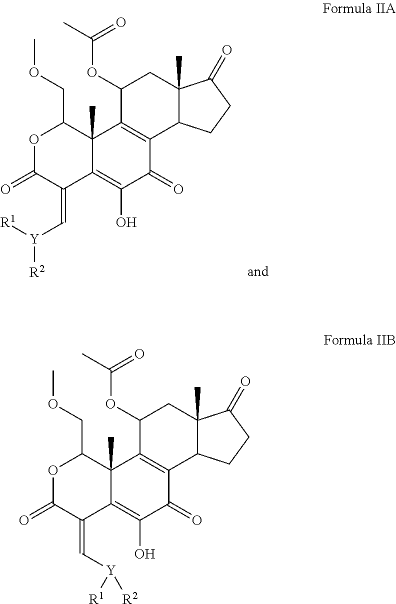

wherein Y is a heteroatom selected from nitrogen and sulfur and R¹ and R² are independently selected from an unsaturated alkyl, cyclic alkyl, or R¹ and R² together with Y form a heterocycle, and cetuximab.

In yet another aspect, provided herein are methods for improving or maintaining the quality of life of a subject diagnosed with a locally advanced, recurrent or metastatic cancer comprising administering to the subject a compound selected from

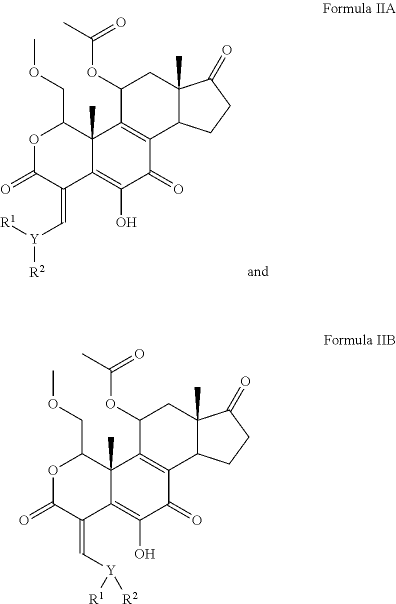

wherein Y is a heteroatom selected from nitrogen and sulfur and R¹ and R² are independently selected from an unsaturated alkyl, cyclic alkyl, or R¹ and R² together with Y form a heterocycle, and cetuximab.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the dosing schedule for continuous dosing of PX-866 in a human clinical trial.

FIG. 2 describes certain patient characteristics in a human clinical trial for testing efficacy of PX-866 in treatment of cancer.

FIG. 3 describes certain adverse events associated with intermittent dosing of PX-866 in a human clinical trial.

FIG. 4 describes certain adverse events associated with continuous dosing of PX-866 in a human clinical trial.

FIG. 5 describes response to intermittent and continuous dosing of PX-866 in a human clinical trial.

FIG. 6 describes certain evaluable patients with stable disease following treatment with PX-866 in a human clinical trial.

FIG. 7 describes pharmacokinetics of PX-866 administration in a human clinical trial.

### DETAILED DESCRIPTION OF THE INVENTION

The PI-3 kinases are a family of related enzymes that are capable of phosphorylating the 3 position hydroxyl group of the inositol ring of phosphatidylinositol. They are linked to a diverse list of cellular functions, including cell growth, proliferation, differentiation, motility, survival and intracellular trafficking Many of these functions relate to the ability of the PI-3 kinases to activate the protein kinase B (Akt). Genetic and pharmacological inactivation of the p110δ isoform of the PI-3 kinase has revealed this enzyme to be important for the function of T cells, B cell, mast cells and neutrophils. Hence, p110δ is considered to be a promising target for drugs that aim to prevent or treat inflammation and autoimmunity and transplant rejection. Recent evidence has shown that the gene encoding the p110α isoform of the PI-3 kinase is mutated in a range of human cancers. For example, mutation of p110α which leads to over-expression of the kinase is found in human lung cancer. PI-3 kinase activity is also found to be elevated in ovarian, head and neck, urinary tract, colon and cervical cancers. Further, a phosphate (PtdIns(3,4,5)P₃) which antagonizes PI-3 kinase activity is absent or mutated in a variety of human cancers, including advanced prostate, endometrial, renal, glial, melanoma, and small cell lung cancers. Thus, inhibition of PI-3 kinase activity provides treatment of certain human cancers.

### Treatment of Cancer

Provided herein, in certain embodiments, are methods for treating cancer in a subject with a combination therapy of a therapeutic and a wortmannin analog. Also provided herein are compounds, pharmaceutical compositions and medicaments comprising such compounds for a combination therapy of a therapeutic and a wortmannin analog. In certain instances, the therapeutic is docetaxel. In other instances, the therapeutic is cetuximab.

Cancers treatable by combination therapies described herein include, but are not limited to, breast cancer, lung cancer, head and neck cancer, brain cancer, abdominal cancer, colon cancer, colorectal cancer, esophageal cancer, parapharyngeal cancer, gastrointestinal cancer, glioma, liver cancer, tongue cancer, neuroblastoma, osteosarcoma, ovarian cancer, renal cancer, pancreatic cancer, retinoblastoma, cervical cancer, uterine cancer, Wilm's tumor, multiple myeloma, skin cancer, lymphoma, leukemia, blood cancer, anaplastic thyroid tumor, sarcoma of the skin, melanoma, adenocystic tumor, hepatoid tumor, non-small cell lung cancer, chondrosarcoma, pancreatic islet cell tumor, prostate cancer including castration resistant forms, ovarian cancer, and/or carcinomas including but not limited to squamous cell carcinoma of the head and neck, colorectal carcinoma, glioblastoma, cervical carcinoma, endometrial carcinoma, gastric carcinoma, pancreatic carcinoma, leiomyosarcoma and breast carcinoma. In some embodiments, the combination therapies described herein treat a lung cancer such as non-small cell lung cancer (NSCLC). In other embodiments, the combination therapies described herein treat a head and neck cancer such as squamous cell carcinoma of the head and neck (SCCHN).

The combination therapies described herein treat various stages of cancer including stages which are locally advanced, metastatic and/or recurrent. In cancer staging, locally advanced is generally defined as cancer that has spread from a localized area to nearby tissues and/or lymph nodes. In the Roman numeral staging system, locally advanced usually is classified in Stage II or III. Cancer which is metastatic is a stage where the cancer spreads throughout the body to distant tissues and organs (stage IV). Cancer designated as recurrent generally is defined as the cancer has recurred, usually after a period of time, after being in remission or after a tumor has visibly been eliminated. Recurrence can either be local, i.e., appearing in the same location as the original, or distant, i.e., appearing in a different part of the body. In certain instances, a cancer treatable by combination therapies described herein is unresectable, or unable to be removed by surgery. In further instances, a cancer treatable by the combination therapies described herein is incurable, i.e., not treatable by current treatment methods.

In some embodiments, the combination therapies described herein are administered as a first-line or primary therapy. Other subjects suitable for treatment by the combination therapies described herein include those that have completed first-line anti-cancer therapy. First-line anti-cancer therapies include chemotherapy, radiotherapy, immunotherapy, gene therapy, hormone therapy, surgery or other therapies that are capable of negatively affecting cancer in a patient, such as for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Chemotherapies for first-line and subsequent therapy include, but are not limited to, hormone modulators, androgen receptor binding agents (e.g., anti-androgens, bicalutamide, flutamide, nilutamide, MDV3100), gonadotropin-releasing hormone agonists and antagonists (e.g., leuprolide, buserelin, histrelin, goserelin, deslorelin, nafarelin, abarelix, cetrorelix, ganirelix degarelix), androgen synthesis inhibitors (abiraterone, TOK-001), temozolomide, mitozolomide, dacarbazine, cisplatin (CDDP), carboplatin, procarbazine, mechlorethamine, cyclophosphamide, camptothecin, ifosfamide, melphalan, chlorambucil, busulfan, nitrosurea, dactinomycin, anthracyclines (e.g., daunorubicin, doxorubicin, epirubicin, idarubicin), bleomycin, plicomycin, mitomycin, etoposide (VP16), tamoxifen, raloxifene, estrogen receptor binding agents, cabazitaxel, paclitaxel, gemcitabine, navelbine, farnesyl-protein transferase inhibitors, transplatinum, 5-fluorouracil, capecitabine, vincristin, vinblastin and methotrexate, topoisomerase inhibitors (e.g., irinotecan, topotecan, camptothecin, etoposide) or any derivative related agent of the foregoing. Many of the above agents are also referred to as hormone therapy agents such as, for example, androgen receptor binding agents, gonadotropin-releasing hormone agonists and antagonists, androgen synthesis inhibitors, estrogen receptor binding agents as well as aromatase inhibitors.

Radiotherapies for first-line and subsequent therapy include factors that cause DNA damage and include what are commonly known as γ-rays, X-rays, and/or the directed delivery of radioisotopes to tumor cells. Other forms of DNA damaging factors include microwaves and UV-irradiation. It is likely that all of these factors affect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

Immunotherapies generally rely on the use of immune effector cells and molecules to target and destroy cancer cells. The immune effector may be, for example, a tumor antigen or an antibody specific for some marker on the surface of a tumor cell. The tumor antigen or antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Alternatively, an tumor antigen may stimulate a subject's immune system to target the specific tumor cells using cytotoxic T cells and NK cells. Immunotherapies include Sipuleucel-T (Provenge®), bevacizumab and the like.

A gene therapy includes a therapeutic polynucleotide is administered before, after, or at the same time as a combination therapy. Therapeutic genes may include an antisense version of an inducer of cellular proliferation (oncogene), an inhibitor of cellular proliferation (tumor suppressor), or an inducer of programmed cell death (pro-apoptotic gene).

Surgery of some type is performed for resectable cancers. Surgery types include preventative, diagnostic or staging, curative and palliative surgery and can be performed as a first-line and subsequent therapy.

In some embodiments, the combination therapies described herein are administered as a second-line therapy after a first-line therapy becomes ineffective or the cancer is recurrent. In other embodiments, the combination therapies described herein administered as a third-line therapy after the first- and second-line therapy fails. In further embodiments, individuals are preselected for having completed a first- or second-line therapy. In some instances, the combination therapies described herein are administered to patients for whom prior platinum-based therapy has failed. In other instances, the combination therapies described herein are administered to patients for whom prior irinotecan therapy has failed.

Subjects, in some embodiments, can also be prescreened or preselected for sensitivity and/or effectiveness of the combination therapies described herein. A subject can be examined for certain biomarkers that allow the subject to be amenable to a combination therapy. For example, biomarkers such as phosphatase and tensin homolog (PTEN) mutations and activating mutations of PI-3K catalytic subunits may increase sensitivity to the combination therapies described herein whereas other mutations such as Ras pathway mutations may decrease sensitivity. In some embodiments, a subject is preselected based on, for example, PTEN mutational status, PTEN copy number, PI3K gene amplification, PI3K catalytic subunit alpha (PIK3CA) mutational status, K-ras mutational status, and/or B-raf mutational status. Additional biomarker candidates are contemplated and described in the below section.

### Phosphatidylinositol-3-Kinases (PI-3Ks)

Phosphatidylinositol-3-kinases (PI-3Ks) are a family of intracellular lipid kinases that play a critical role in transmitting signals from cell surface receptors on the plasma membrane to downstream signaling intermediates. PI-3Ks are linked to a diverse list of cellular functions, including cell growth, proliferation, differentiation, motility, survival and intracellular trafficking There are 3 classes of PI-3K (Class I, II and III) which are classified based upon their structure and substrate specificity. Class I PI-3K are heterodimers formed by a regulatory subunit and a catalytic p110 subunit that phosphorylate membrane-associated phosphatidylinositol 4,5-bisphosphate (PIP2) to form phosphatidylinositol 3,4,5-trisphosphate (PIP3). PIP3 binds to the serine protein kinase AKT, which is reportedly the primary effector of PI-3K, triggering activation of downstream signaling intermediates, including mammalian target of rapamycin (mTOR), with subsequent effects on cell growth and metabolism, survival, and proliferation, as well as angiogenesis. The tumor suppressor gene phosphatase and tensin homolog (PTEN) reportedly counteracts the activity of Class I PI-3K by dephosphorylating PIP3 back to PIP2. PI-3K activation reportedly affects other AKT-independent pathways including Bruton tyrosine kinase and Tec family kinases, serum and glucocorticoid regulated kinases, and regulators of GTPases, although the role of these pathways is less well defined.

Class I PI-3K is further divided into Class I_{A} and Class I_{B} subfamilies. Class I_{A} PI-3K are formed by a regulatory p85 subunit (PIK3R1) and a catalytic p110 subunit that are primarily activated by receptor tyrosine kinases such as epidermal growth factor receptor (EGFR), insulin-like growth factor (IGF), platelet-derived growth factor (PDGF) and Her2/neu. Several isoforms exist for each subunit, including α, β, γ and δ isoforms of p110. The α and β isoforms are expressed ubiquitously, whereas expression of the δ isoform is restricted to leukocytes. Class I_{B} PI-3K are composed of a p110 subunit and a p101 regulatory subunit. Class I_{B} PI-3K are activated by G protein-coupled receptors. The best characterized Class I_{B} PI-3K contains the gamma isoform of p110, and is expressed primarily in leukocytes, as well as heart, pancreas, skeletal muscle, and liver.

Increased signaling through Class I_{A} PI-3Ks has been implicated in many different forms of cancer. Cancers in which PI-3K pathway abnormalities have been identified include non-small cell lung cancer (NSCLC), breast carcinoma, ovarian carcinoma, endometrial carcinoma, prostate carcinoma, squamous cell carcinoma of the head and neck (SCCHN), cervical cancer, glioblastoma, melanoma, and colorectal carcinoma. PI-3Ks are also contemplated in other cancers. Reported mechanisms which lead to increased signaling through the PI-3K pathway include increased receptor tyrosine kinase (RTK) activity, activating mutations in the p110α isoform, mutations in the p85 subunit, and mutations and deletions in PTEN. Amplification of the PIK3CA gene has also been observed in a number of tumors, including squamous cell carcinomas of the lung and head and neck, although this observation has not yet been linked directly to increased PI-3K activity.

### Wortmannin Analogs

Wortmannin is a naturally occurring compound isolated from culture broths of fungal strains, Penicillium wortmannin, Talaromyces wortmannin, Penicillium Funiculosum and related micro-organisms. Wortmannin irreversibly inhibits PI-3K through covalent interaction with a specific lysine on the kinase: Lys⁸⁰² of the ATP binding pocket of the catalytic site of the p110α isoform or Lys⁸⁸³ of the p110γ isoform. Most isoforms of PI-3K, such as p110α, p1101β, p110δ and p110γ for example, are inhibited equally by wortmannin. Wortmannin demonstrates liver and hematologic toxicity, however, and is a biologically unstable molecule. Samples stored as aqueous solutions at either 37° C. or 0° C. at neutral pH are subject to decomposition by hydrolytic opening of the furan ring. It has been shown that the electrophilicity of the furan ring is central to the inhibitory activity of wortmannin. The irreversible inhibition of PI-3K occurs by formation of an enamine following the attack of the active lysine of the kinase on the furan ring at position C(20) of wortmannin. Decomposition of wortmannin interferes with its inhibitory activity on PI-3Ks. Although wortmannin is a nanomolar inhibitor of PI-3K, its instability and toxicity to the liver results in variable activity in animal models. Wortmannin analogs have been contemplated and described that improve toxicity and stability of the base wortmannin compound.

In some embodiments, wortmannin analogs suitable for combination therapies described herein include compounds of Formula IA or IB:

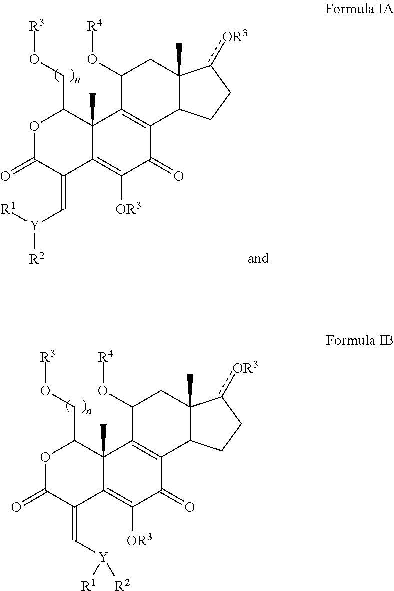

wherein:
— is an optional bond;
n is 1-6;
Y is a heteroatom;
R¹ and R² are independently selected from an unsaturated alkyl, non-linear alkyl, cyclic alkyl, and substituted alkyl or R¹ and R² together with the atom to which they are attached form a heterocycloalkyl group;
R³ is absent, H, or C₁-C₆ substituted or unsubstituted alkyl;
R⁴ is (C═O)R⁵, (C═O)OR⁵, (S═O)R⁵, (SO₂)R⁵, (PO₃)R⁵, (C═O)NR⁵R⁶;
R⁵ is substituted or unsubstituted C₁-C₆ alkyl; and
R⁶ is substituted or unsubstituted C₁-C₆ alkyl.

In some embodiments, wortmannin analogs suitable for combination therapies described herein include compounds of Formula IIA or IIB:

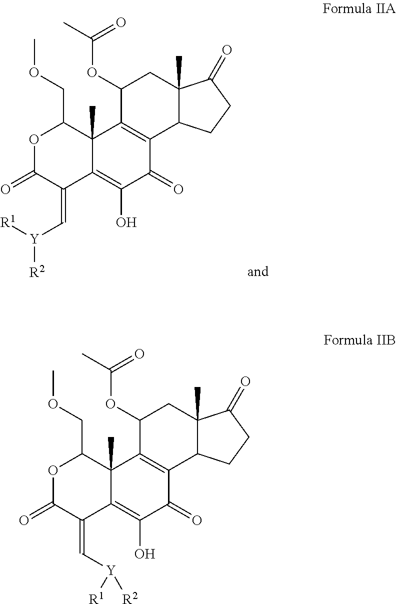

wherein Y is a heteroatom and R¹ and R² are independently selected from an unsaturated alkyl, non-linear alkyl, cyclic alkyl, and substituted alkyl or R¹ and R² together with Y form a heterocycle.

In certain embodiments of compounds of formula IIA or IIB, Y is a heteroatom selected from nitrogen and sulfur and R¹ and R² are independently selected from an unsaturated alkyl, cyclic alkyl, or R₁ and R₂ together with Y form a heterocycle.

In further embodiments, a wortmannin analog is Acetic acid 4-diallylaminomethylene-6-hydroxy-1-α-methoxymethyl-10β,13β-dimethyl-3,7,17-trioxo-1,3,4,7,10,11β,12,13,14α,15,16,17-dodecahydro-2-oxa-cyclopenta[α]phenanthren-11-yl ester (PX-866) having the structure,

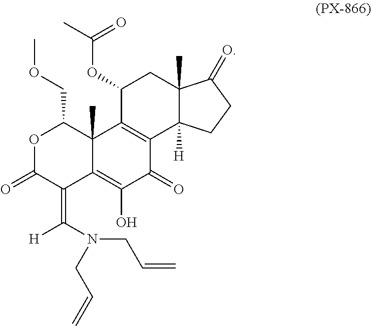 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

In yet further embodiments, a wortmannin analog is Acetic acid 6-hydroxy-1α-methoxymethyl-10β,13β-dimethyl-3,7,17-trioxo-4-pyrrolidin-1-methylene-1,3,4,7,10, 11β,12,13,14α,15,16,17-dodecahydro-2-oxa-cyclopenta[α]phenanthren-11-yl (PX-867) having the structure,

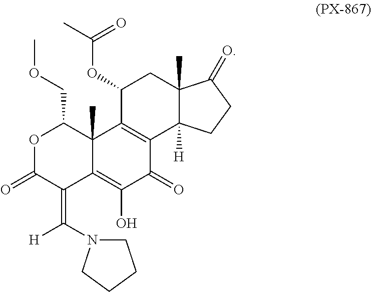 [SMILES: COC[C@H]1OC(=O)/C(=C/N2CCCC2)C2=C(O)C(=O)C3=C([C@H](OC(C)=O)C[C@]4(C)C(=O)CC[C@@H]34)[C@]21C]

In additional embodiments, wortmannin analogs suitable for combination therapies described herein include compounds selected from, but not limited to, PX-868, PX-870, PX-871, PX-880, PX-881, PX-882, PX-889, PX-890, DJM2-170, DJM2-171, DJM2-177, DJM2-181 and combinations thereof. In some embodiments, wortmannin analogs suitable for combination therapies described herein include compounds described in GB Pat. No. 2302021, which compounds are incorporated herein by reference.

### Further Forms of Wortmannin Analogs

In the scope of the embodiments, wortmannin analogs include further forms of the compounds described herein such as pharmaceutically acceptable salts, solvates (including hydrates), amorphous phases, partially crystalline and crystalline forms (including all polymorphs), prodrugs, metabolites, N-oxides, isotopically-labeled and stereo-isomers. Wortmannin analogs can be prepared as a pharmaceutically acceptable salts formed when an acidic proton present in the parent compound either is replaced by a metal ion, for example an alkali metal ion, an alkaline earth ion, or an aluminum ion; or coordinates with an organic base. In addition, the salt forms of the disclosed compounds can be prepared using salts of the starting materials or intermediates.

In some of the embodiments described herein, wortmannin analogs can be prepared as a pharmaceutically acceptable acid addition salt (which is a type of a pharmaceutically acceptable salt) by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Alternatively, in some of the embodiments described herein, wortmannin analogs can be prepared as a pharmaceutically acceptable base addition salts (which is a type of a pharmaceutically acceptable salt) by reacting the free acid form of the compound with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of wortmannin analogs can be conveniently prepared or formed during the processes described herein. By way of example only, hydrates of wortmannin analogs can be conveniently prepared by recrystallization from an aqueous/organic solvent mixture, using organic solvents including, but not limited to, dioxane, toluene, alkyl acetate, anisole, tetrahydrofuran or methanol. In addition, the compounds provided herein can exist in unsolvated as well as solvated forms. In general, the solvated forms are considered equivalent to the unsolvated forms for the purposes of the compounds and methods provided herein.

In some of the embodiments described herein, wortmannin analogs include crystalline forms, also known as polymorphs. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

In some of the embodiments described herein, wortmannin analogs in unoxidized form can be prepared from N-oxides of compounds of Formula (1) by treating with a reducing agent, such as, but not limited to, sulfur, sulfur dioxide, triphenyl phosphine, lithium borohydride, sodium borohydride, phosphorus trichloride, tribromide, or the like in a suitable inert organic solvent, such as, but not limited to, acetonitrile, ethanol, aqueous dioxane, or the like at 0 to 80° C.

In some embodiments, wortmannin analogs are isotopically-labeled, which are identical to those recited in the various formulae and structures presented herein, but for the fact that one or more atoms are replaced by an atom having an atomic mass or mass number different from the atomic mass or mass number usually found in nature. In some embodiments, one or more hydrogen atoms are replaced with deuterium. In some embodiments, metabolic sites on the compounds described herein are deuterated. In some embodiments, substitution with deuterium affords certain therapeutic advantages resulting from greater metabolic stability, such as, for example, increased in vivo half-life or reduced dosage requirements.

In some of the embodiments described herein, wortmannin analogs can be prepared as prodrugs. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a wortmannin analog which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety.

In some of the embodiments described herein, wortmannin analogs are metabolites. A “metabolite” of a wortmannin analog disclosed herein is a derivative of that wortmannin analog that is formed when the wortmannin analog is metabolized. The term “active metabolite” refers to a biologically active derivative of a wortmannin analog that is formed when the wortmannin analog is metabolized (biotransformed). The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a wortmannin analog. For example, cytochrome P450 catalyzes a variety of oxidative and reductive reactions while uridine diphosphate glucuronyltransferases (UGT) catalyze the transfer of an activated glucuronic-acid molecule to aromatic alcohols, aliphatic alcohols, carboxylic acids, amines and free sulphydryl groups (e.g. conjugation reactions). Further information on metabolism is available in The Pharmacological Basis of Therapeutics, 9th Edition, McGraw-Hill (1996). In one embodiment, metabolites of the compounds disclosed herein are identified either by administration of compounds to a host and analysis of tissue samples from the host, or by incubation of compounds with hepatic cells in vitro and analysis of the resulting compounds.

Metabolites of wortmannin analogs, in some embodiments described herein, include, but are not limited to, metabolites resulting from first pass metabolism. In some embodiments, the metabolite is a 17-hydroxy (17-OH) derivative of a wortmannin analog. In some embodiments, the metabolite is a derivative of PX-866. In other embodiments, the metabolite is a derivative of PX-867.

In some instances a metabolite of PX-866 has the following structural formula:

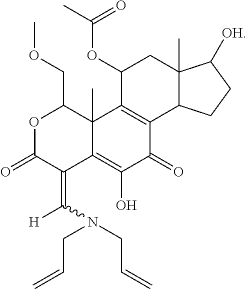 [SMILES: C=CCN(C=C1C(=O)OC(COC)C2(C)C1=C(O)C(=O)C1=C2C(OC(C)=O)CC2(C)C(O)CCC12)CC=C]

In other instances a metabolite of PX-867 has the following structural formula:

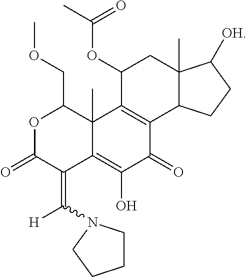 [SMILES: COCC1OC(=O)C(=CN2CCCC2)C2=C(O)C(=O)C3=C(C(OC(C)=O)CC4(C)C(O)CCC34)C21C]

In further embodiments, a metabolite of a wortmannin analog is a 11,17-hydroxy (11,17-OH) derivative of a wortmannin analog.

In some instances a metabolite of PX-866 has the following structural formula:

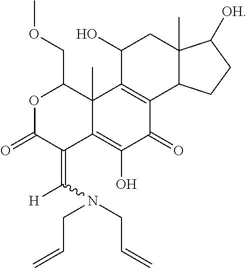 [SMILES: C=CCN(C=C1C(=O)OC(COC)C2(C)C1=C(O)C(=O)C1=C2C(O)CC2(C)C(O)CCC12)CC=C]

In other instances a metabolite of PX-867 has the following structural formula:

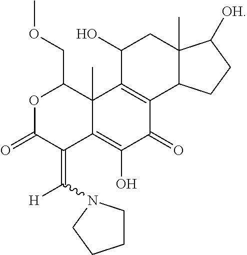 [SMILES: COCC1OC(=O)C(=CN2CCCC2)C2=C(O)C(=O)C3=C(C(O)CC4(C)C(O)CCC34)C21C]

PX-866 is an pan-isoform inhibitor of Class I P1-3K that covalently binds to ATP binding site of the p110 catalytic subunit. Described herein are studies that illustrate rapid metabolism of PX-866 to a 17-hydroxy PX-866 derivative. The 17-hydroxy PX-866 metabolite has a 2-5 fold increase in potency in cell proliferation assays versus p110α and p110β isoforms. For example, in cell based assays, potency of the 17-hydroxy metabolite is p110α IC50 14 nM vs 39 nM for the parent compound (PX-866), potency of the 17-hydroxy metabolite is p110β IC50 57 nM vs. 88 nM for the parent compound (PX-866).

Table 1 illustrates the potency of 17-hydroxy PX-866 metabolite in in vitro kinase assays:

IC50 nM

| Target | PX-866 | 17-OH PX-866 |
|---|---|---|
| PIK3CA | 39 | 14 |
| PIK3CB | 88 | 57 |
| PIK3CD | 124 | 131 |
| PIK3CG | 198 | 148 |

### Synthesis of Wortmannin Analogs

Wortmannin analogs described herein may be synthesized using standard synthetic techniques known to those of skill in the art or using methods known in the art in combination with methods described herein. In additions, solvents, temperatures and other reaction conditions presented herein may vary according to the practice and knowledge of those of skill in the art.

The starting material used for the synthesis of wortmannin analogs described herein can be obtained from commercial sources, such as Aldrich Chemical Co. (Milwaukee, Wis.), Sigma Chemical Co. (St. Louis, Mo.), or the starting materials can be synthesized. The wortmannin analogs described herein, and other related compounds having different substituents can be synthesized using techniques and materials known to those of skill in the art, such as described, for example, in March, ADVANCED ORGANIC CHEMISTRY 4th Ed., (Wiley 1992); Carey and Sundberg, ADVANCED ORGANIC CHEMISTRY 4th Ed., Vols. A and B (Plenum 2000, 2001), and Green and Wuts, PROTECTIVE GROUPS IN ORGANIC SYNTHESIS 3rd Ed., (Wiley 1999) (all of which are incorporated by reference in their entirety). General methods for the preparation of wortmannin analogs as disclosed herein may be derived from known reactions in the field, and the reactions may be modified by the use of appropriate reagents and conditions, as would be recognized by the skilled person, for the introduction of the various moieties found in the formulae as provided herein.

Additional synthesis methods and schemes for the wortmannin analogs described herein can be found in, for example, U.S. Pat. No. 5,480,906, U.S. Pat. No. 7,335,679, and U.S. Patent Appl. Pub. No. 2007/0191466, each of which is incorporated herein by reference for synthesis of wortmannin analogs.

### Pharmaceutical Compositions of Wortmannin Analogs

Pharmaceutical compositions containing wortmannin analogs can be administered in therapeutically effective amounts as pharmaceutical compositions by any conventional form and route known in the art including, but not limited to: injection, transdermal, nasal, pulmonary, vaginal, rectal, buccal, ocular, otic, local, topical, or oral administration. In certain embodiments, an injectable pharmaceutical composition of a wortmannin analog is an intramuscular, intravenous, subcutaneous, intranodal, intratumoral, intracisternal, intraperitoneal, or intradermal injection. In addition, the pharmaceutical composition containing wortmannin analogs may be provided in the form of a rapid release formulation, in the form of an extended release formulation, or in the form of an intermediate release formulation.

For oral administration, wortmannin analogs can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a patient to be treated.

Pharmaceutical preparations for oral use can be obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents may be added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. In some embodiments, a wortmannin analog is in powder form and is directly filled into hard gelatin capsules.

For buccal or sublingual administration, the compositions may take the form of tablets, lozenges, or gels formulated in conventional manner.

Injectable compositions may involve for bolus injection or continuous infusion. An injectable composition of wortmannin analogs may be in a form suitable for parenteral or any other type of injection as a sterile suspensions, solutions or emulsions in oily or aqueous vehicles, and may contain formulatory agents such as suspending, stabilizing and/or dispersing agents. The composition may be formulated for intramuscular, intravenous, subcutaneous, intranodal, intratumoral, intracisternal, intraperitoneal, and/or intradermal injection. Pharmaceutical formulations for injection administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

In various embodiments, wortmannin analog compositions are in liquid form for ocular or otic delivery. Liquid forms include, by way of non-limiting example, neat liquids, solutions, suspensions, dispersions, colloids, foams and the like and can be formulated by known methods.

Wortmannin analogs can be administered topically and can be formulated into a variety of topically administrable compositions, such as solutions, suspensions, lotions, gels, pastes, medicated sticks, balms, creams or ointments. Such pharmaceutical compounds can contain solubilizers, stabilizers, tonicity enhancing agents, buffers and preservatives.

Formulations suitable for transdermal administration of wortmannin analogs may employ transdermal delivery devices and transdermal delivery patches and can be lipophilic emulsions or buffered, aqueous solutions, dissolved and/or dispersed in a polymer or an adhesive. Such patches may be constructed for continuous, pulsatile, or on demand delivery of pharmaceutical agents. Still further, transdermal delivery of the wortmannin analogs can be accomplished by means of iontophoretic patches and the like. Additionally, transdermal patches can provide controlled delivery of the wortmannin analogs. The rate of absorption can be slowed by using rate-controlling membranes or by trapping the compound within a polymer matrix or gel. Conversely, absorption enhancers can be used to increase absorption. An absorption enhancer or carrier can include absorbable pharmaceutically acceptable solvents to assist passage through the skin. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

For administration by inhalation for pulmonary or nasal delivery, wortmannin analogs maybe in a form as an aerosol, a mist or a powder. Pharmaceutical compositions of wortmannin analogs are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

Wortmannin analogs may also be formulated in rectal or vaginal compositions such as enemas, douches, gels, foams, aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

One may administer wortmannin analogs in a local rather than systemic manner, for example, via injection of the compound directly into an organ, often in a depot or sustained release formulation. Furthermore, one may administer pharmaceutical composition containing wortmannin analogs in a targeted drug delivery system, for example, in a liposome coated with organ-specific antibody. The liposomes will be targeted to and taken up selectively by the organ. Pharmaceutical compositions of wortmannin analogs may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions comprising a wortmannin analogs may be manufactured in a conventional manner, such as, by way of example only, by means of conventional mixing, dissolving, granulating, dragee-making, levigating, emulsifying, encapsulating, entrapping or compression processes.

The pharmaceutical compositions will include at least one pharmaceutically acceptable carrier, diluent or excipient and a wortmannin analog described herein as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these compounds having the same type of activity. In some situations, wortmannin analogs may exist as tautomers. All tautomers are included within the scope of the compounds presented herein. Additionally, wortmannin analogs described herein can exist in unsolvated as well as solvated forms with pharmaceutically acceptable solvents such as water, ethanol, and the like. The solvated forms of wortmannin analogs presented herein are also considered to be disclosed herein. In addition, the pharmaceutical compositions may include other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions can also contain other therapeutically valuable substances.

Methods for the preparation of compositions comprising wortmannin analogs described herein include formulating the wortmannin analogs with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

Further forms of pharmaceutical compositions of wortmannin analogs can be integrated with other active agents, e.g., docetaxel, in a unitary dosage form for combination therapies. The unitary dosage forms can be formulated to release where both agents are released simultaneously or where there is sequential release of each agent via known modified release mechanisms including but not limited to timed release, delayed release, pH release, pulsatile release and the like.

A summary of pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference in their entirety.

### Wortmannin Analogs Dosages

Dosages of wortmannin analogs described herein (e.g., compounds of Formula IA, IB, IIA or IIB or any other PI-3 kinase inhibitor and/or wortmannin analog described herein) can be determined by any suitable method. Maximum tolerated doses (MTD) and maximum response doses (MRD) can be determined via established animal and human experimental protocols as well as in the examples described herein. For example, toxicity and therapeutic efficacy of wortmannin analogs can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Wortmannin analogs exhibiting high therapeutic indices are of interest. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given wortmannin analog that corresponds to such an amount varies depending upon factors such as the particular compound, disease condition and its severity, the identity (e.g., weight, sex) of the subject or host in need of treatment, but can nevertheless be determined according to the particular circumstances surrounding the case, including, e.g., the specific agent being administered, the route of administration, the condition being treated, and the subject or host being treated.

In other embodiments, however, doses employed for adult human treatment are typically in the range of about 0.01 mg to about 5000 mg per day, or about 1 mg to about 1500 mg per day. In one embodiment, the desired dose is conveniently presented in a single dose or in divided doses administered simultaneously (or over a short period of time) or at appropriate intervals, for example as two, three, four or more sub-doses per day.

In some embodiments, wortmannin analogs are provided in a dose per day from about 0.01 mg to 1000 mg, from about 0.1 mg to about 100 mg, from about 1 to about 20, from about 2 mg to about 12 mg. In certain embodiments, wortmannin analogs are provided in a daily dose of about 0.01 mg, about 0.05 mg, about 0.1 mg, about 0.2 mg, about 0.4 mg, about 0.6 mg, about 0.8 mg, about 1 mg, about 1.5 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 15 mg, about 20 mg, about 25 mg, about 30 mg, about 40 mg, about 50 mg, about 75 mg, about 100 mg, about 150 mg, about 200 mg, about 250 mg, about 300 mg, about 500, mg, about 750 mg, about 1000 mg, or more, or any range derivable therein. In certain instances, wortmannin analogs are provided in a dose per day of about 1 mg. In certain instances, wortmannin analogs are provided in a dose per day of about 2 mg. In certain instances, wortmannin analogs are provided in a dose per day of about 3 mg. In certain instances, wortmannin analogs are provided in a dose per day of about 4 mg. In certain instances, wortmannin analogs are provided in a dose per day of about 5 mg. In certain instances, wortmannin analogs are provided in a dose per day of about 6 mg. In certain instances, wortmannin analogs are provided in a dose per day of about 7 mg. In certain instances, wortmannin analogs are provided in a dose per day of about 8 mg. In certain instances, wortmannin analogs are provided in a dose per day of about 9 mg. In certain instances, wortmannin analogs are provided in a dose per day of about 10 mg. In certain instances, wortmannin analogs are provided in a dose per day of about 11 mg. In certain instances, wortmannin analogs are provided in a dose per day of about 12 mg. The dose per day described herein can be given once per day or multiple times per day in the form of sub-doses given b.i.d., t.i.d., q.i.d., or the like where the number of sub-doses equal the dose per day.

In further embodiments, the daily dosages appropriate for the compound of Formula IA, IB, IIA or IIB or any other PI-3 kinase inhibitor and/or wortmannin analog described herein are from about 0.001 to about 100 mg/kg per body weight. In one embodiment, the daily dosages appropriate for the compound of Formula IA, IB, IIA or JIB or any other PI-3 kinase inhibitor and/or wortmannin analog described herein are from about 0.01 to about 10 mg/kg per body weight. In some embodiments, an indicated daily dosage in a large mammal, including, but not limited to, humans, is in the range from about 0.02 mg to about 1000 mg, conveniently administered in divided doses, including, but not limited to, up to four times a day. In one embodiment, the daily dosage is administered in extended release form. In certain embodiments, suitable unit dosage forms for oral administration comprise from about 1 to 500 mg active ingredient. In other embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In other embodiments wortmannin analogs are provided at the maximum tolerated dose (MTD). In other embodiments, the amount of wortmannin analogs administered is from about 10% to about 90% of the maximum tolerated dose (MTD), from about 25% to about 75% of the MTD, or about 50% of the MTD. In particular embodiments, the amount of wortmannin analogs administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD.

### Docetaxel

A number of microtubule inhibitors or spindle poisons such as taxoids (e.g., docetaxel, paclitaxel, cabazitaxel), vinca alkaloids (e.g., vincristine, vinorelbine, vinflunine) and epothilones (e.g., epothilone A-F, ixabepilone) are used clinically as standard anti-neoplastic agents. Among these, docetaxel (Taxotere®), is an agent that is often used as a single agent as part of standard of care following first line therapy in the management of patients with recurrent or metastatic cancers including non-small cell lung cancer, castration-resistant prostate cancer, squamous cell carcinoma of the head and neck, breast carcinoma and ovarian carcinoma. While docetaxel is associated with some clinical benefit in all of these conditions based on response rate (RECIST criteria or Prostate Specific Antigen, PSA decline), progression-free survival (PFS) or overall survival, the benefit is often small and many, if not all, patients ultimately progress.

Docetaxel is prepared semisynthesis beginning with a precursor extracted from renewable needle biomass of yew trees. The chemical name of docetaxel is (2R,3S)—N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate. Docetaxel has the following structural formula:

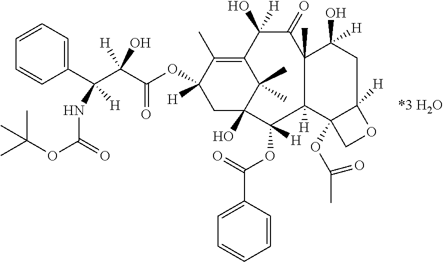

Docetaxel is disclosed in U.S. Pat. Nos. 4,814,470; 6,197,980; 6,838,569 and 6,022,985, which is incorporated by reference herein for the preparation, synthesis and formulations thereof.

It is contemplated that docetaxel acts by disrupting the microtubular network in cells that is essential for mitotic and interphase cellular functions. Docetaxel reportedly binds to free tubulin and promotes the assembly of tubulin into stable microtubules while simultaneously inhibiting their disassembly. The production of microtubule bundles without normal function and the stabilization of microtubules are thought to result in the inhibition of mitosis in cells. One of docetaxel's properties is that its binding to microtubules does not alter the number of protofilaments in the bound microtubules, a feature which differs from most other microtubule inhibitors currently in clinical use.

Derivatives, polymorphs, other salts, solvates and forms of docetaxel are contemplated in the embodiments of the combination therapies described herein. Other derivatives, polymorphs and forms of docetaxel are described in U.S. Pat. No. 5,476,954; U.S. Pat. Appl. Pub. No. 2005/0065138; U.S. Pat. Appl. Pub. No. 2004/0138142; U.S. Pat. Appl. Pub. No. 2010/0160653 and U.S. Pat. Appl. Pub. No. 2010/0197944, each of which is incorporated herein by reference for derivatives, polymorphs, other salts, solvates and other forms of docetaxel. Other taxoids including paclitaxel are within the scope of the embodiments described herein.

Docetaxel is administered by any known method. In some embodiments, docetaxel is administered by intravenous infusion. In other embodiments, docetaxel is administered orally. For intravenous infusion administration of docetaxel, the dosage is determined by a medical practitioner who evaluates the treatment to be sought, patient condition and other factors such as side effects. In some embodiments, dosages for intravenous infusion administration of docetaxel range from about 20 mg/m² to about 200 mg/m², about 35 mg/m² to about 150 mg/m², or about 50 mg/m² to about 100 mg/m² over a period of about one hour. In other embodiments, dosages for intravenous infusion administration of docetaxel are about 20 mg/m², about 30 mg/m², about 40 mg/m², about 45 mg/m², about 50 mg/m², about 55 mg/m², about 60 mg/m², about 65 mg/m², about 70 mg/m², about 75 mg/m², about 80 mg/m², about 90 mg/m², about 100 mg/m², about 110 mg/m², about 120 mg/m², about 130 mg/m², about 140 mg/m², about 150 mg/m², about 160 mg/m², about 170 mg/m², about 180 mg/m², about 190 mg/m² or about 200 mg/m² over a period of about one hour. In certain instances, intravenous infusion administration of docetaxel is dosed at about 55 mg/m². In other instances, intravenous infusion administration of docetaxel is dosed at about 60 mg/m². In yet other instances, intravenous infusion administration of docetaxel is dosed at about 75 mg/m². In further instances, intravenous infusion administration of docetaxel is dosed at about 100 mg/m².

In other embodiments docetaxel is provided at the maximum tolerated dose (MTD). In other aspects, the amount of docetaxel administered is from about 10% to about 90% of the maximum tolerated dose (MTD), from about 25% to about 75% of the MTD, or about 50% of the MTD. In particular embodiments, the amount of docetaxel administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD.

### EGFR Inhibitor—Cetuximab

The epidermal growth factor receptor (EGFR, HER1, c-ErB-1) is a transmembrane glycoprotein that is a member of a subfamily of type I receptor tyrosine kinases including EGFR, HER2, HER3 and HER4. The EGFR is widely expressed in many epithethial tissues, including skin and hair follicles. Expression of EGFR is also detected in many human cancers including those of the head and neck, colon and rectum.

A number of epidermal growth factor receptor (EGFR) inhibitors including antibodies (e.g., cetuximab, panitumumab, zalutumumab, nimotuzumab, trastuzumab, pertuzumab and matuzumab) as well as small molecules (e.g., gefitinib, erlotinib, lapatinib) have been developed and some are used clinically as standard anti-neoplastic agents. Among these, cetuximab (Erbitux®, ImClone Systems, NY), is an agent that is often used as a single agent as part of standard of care following first line therapy in the management of patients with recurrent or metastatic cancers including squamous cell carcinoma of the head and neck and colorectal carcinoma. Cetuximab has also been used in combination with radiation therapy or with a topoisomerase inhibitor (e.g., irinotecan).

Cetuximab is a chimeric mouse-human monoclonal antibody (MAb) that binds the EGFR in its extracellular domain. In in vitro cell line studies, cetuximab reportedly blocks Epidermal Growth Factor-induced autophosphorylation of the EGFR, induces dimerization and downregulation of the EGFR and perturbs cell cycle progression by inducing a G1 arrest through an increase in protein levels of p27kip1 inihibitor of cyclin-dependent kinases. In further cell line studies, cetuximab reportedly inhibits tumor-induced angiogenesis. Cetuximab has also demonstrated preclinical activity in vitro and in vivo, as a single agent and in combination with cytotoxic agents and radiotherapy in a number of human cancer cell lines, including colorectal, pancreatic, prostate, breast, head and neck, glioma and ovarian cancer, to name a few.

Cetuximab is currently approved for use in combination with irinotecan in EGFR-expressing metastatic colorectal cancers refractory to irinotecan and as single agent in EGFR-expressing metastatic colorectal cancers after failure of both irinotecan and oxaliplatin-based regimens or in patients intolerant of irinotecan-based regimens. In addition to colorectal cancers, cetuximab is also approved for treatment of locally or regionally advanced squamous cell carcinoma of the head and neck (SCCHN) in combination with radiation therapy, and as a single agent for recurrent or metastatic SCCHN progressing after platinum-based therapy.

While cetuximab is a successful therapy for subjects with various cancers, treatment benefits have not been observed in subjects with certain Kras mutations such as mutations in codons 12 or 13 that result in constitutive activation of the Kras protein. It is contemplated that because Kras normally is a downstream mediator of EGFR signaling from extracellular signals, an EGFR inhibitor, such as cetuximab, is ineffective in cancers with Kras mutations that are EGFR independent.

Cetuximab is disclosed in U.S. Pat. No. 6,217,886 and Goldstein et al., Cin. Cancer Res., 1995, 1: 1311-1318, each of which is incorporated by reference herein for the preparation, synthesis and formulations thereof. Further formulations and preparations of cetuximab are described in U.S. Pat. Appl. Nos. 2007/0172475 and 2007/0122411, each incorporated by reference for the cetuximab formulations and preparations thereof. Other EGFR inhibitors including panitumumab, zalutumumab, nimotuzumab, trastuzumab, pertuzumab, matuzumab, gefitinib, erlotinib and lapatinib are within the scope of the embodiments described herein.

Cetuximab is administered by any known method. In some embodiments, cetuximab is administered by intravenous infusion. For intravenous infusion administration of cetuximab, the dosage is determined by a medical practitioner who evaluates the treatment to be sought, patient condition and other factors such as side effects. In some embodiments, dosages for intravenous infusion administration of cetuximab range from about 50 mg/m² to about 600 mg/m², about 100 mg/m² to about 500 mg/m², or about 200 mg/m² to about 400 mg/m² over a period of about one to two hours. In other embodiments, dosages for intravenous infusion administration of cetuximab are about 50 mg/m², about 75 mg/m², about 100 mg/m², about 125 mg/m², about 150 mg/m², about 175 mg/m², about 200 mg/m², about 250 mg/m², about 275 mg/m², about 300 mg/m², about 325 mg/m², about 350 mg/m², about 375 mg/m², about 400 mg/m², about 425 mg/m², about 450 mg/m², about 475 mg/m², about 500 mg/m², about 525 mg/m², about 550 mg/m², about 575 mg/m², or about 600 mg/m² over a period of about one or two hours. In certain instances, intravenous infusion administration of cetuximab is dosed at about 400 mg/m². In other instances, intravenous infusion administration of cetuximab is dosed at about 250 mg/m². In yet other instances, intravenous infusion administration of cetuximab is dosed at about 200 mg/m². In further instances, intravenous infusion administration of cetuximab is dosed at about 100 mg/m².

In any of the previous embodiments, the infusion administration rate is about 10 mg/minute, about 9 mg/minute, about 8 mg/minute, about 7 mg/minute, about 6 mg/minute, about 5 mg/minute, about 4 mg/minute, about 3 mg/minute, about 2 mg/minute or about 1 mg/minute. In some instances, the infusion administration rate is about 10 mg/minute. In other instances, the infusion administration rate is about 8 mg/minute. In other instances, the infusion administration rate is about 6 mg/minute.

In other embodiments cetuximab is provided at the maximum tolerated dose (MTD). In other aspects, the amount of cetuximab administered is from about 10% to about 90% of the maximum tolerated dose (MTD), from about 25% to about 75% of the MTD, or about 50% of the MTD. In particular embodiments, the amount of cetuximab administered is from about 5%, 10%, 15%, 20%, 25%, 30%, 35%, 40%, 45%, 50%, 55%, 60%, 65%, 70%, 75%, 80%, 85%, 90%, 95%, 99%, or higher, or any range derivable therein, of the MTD.

### Administration of a Wortmannin Analog Combination Therapy

Administration of a wortmannin analog in combination with another therapeutic are at dosages and compositions described herein or at other dose levels and compositions determined and contemplated by a medical practitioner.

In certain embodiments, the combination therapies described herein are administered for prophylactic and/or therapeutic treatments. In certain therapeutic applications, a combination therapy is administered to a patient already suffering from a cancer, in an amount sufficient to cure or at least partially arrest the symptoms of the cancer. Amounts effective for this use depend on the severity and course of the cancer, previous therapy, the patient's health status, weight, and response to the drugs, and the judgment of the treating physician. Therapeutically effective amounts of wortmannin analog or the other therapeutic are optionally determined by methods including, but not limited to, a dose escalation clinical trial, such as described in Example 1.

In prophylactic applications, a combination therapy described herein are administered to a patient susceptible to or otherwise at risk of a particular cancer. Such an amount is defined to be a “prophylactically effective amount or dose.” In this use, the precise amounts also depend on the patient's state of health, weight, and the like. When used in a patient, effective amounts for this use will depend on the severity and course of the cancer, previous therapy, the patient's health status and response to the drugs, and the judgment of the treating physician.

In certain embodiments wherein the patient's condition does not improve, upon the doctor's discretion the administration of the compounds in a combination therapy are administered chronically, that is, for an extended period of time, including throughout the duration of the patient's life in order to ameliorate or otherwise control or limit the symptoms of the patient's cancer. In other embodiments, administration of a combination therapy continues until complete or partial response of a cancer.

When administered as an intravenous infusion or by other known methods, a therapeutic in a combination therapy such as docetaxel or cetuximab is given to a subject periodically, where each period is referred to as a treatment cycle. Docetaxel or cetuximab administration periods include, but are not limited to, once every 3 days, once every 7 days, once every 10 days, once every 2 weeks, once every 3 weeks, once every 4 weeks, once every 5 weeks, once every 6 weeks or more. In some embodiments, the therapeutic is administered once every 3 weeks. Treatment cycles also include, but are not limited to 1 cycle, 2 cycles, 3 cycles, 4 cycles, 5 cycles, 6 cycles, 7 cycles, 8 cycles, 9 cycles, 10 cycles, 11 cycles, 12 cycles, 13 cycles, 14 cycles, 15 cycles, 16 cycles, 17 cycles, 18 cycles, 19 cycles, 20 cycles, 25 cycles, 30 cycles, 40 cycles, or more. For an administration period of once every three weeks, the above representative cycles would last 3 weeks, 6 weeks, 9 weeks, 12 weeks, 15 weeks, 18 weeks, 21 weeks, 24 weeks, 27 weeks, 30 weeks, 33 weeks, 36 weeks, 39 weeks, 42 weeks, 45 weeks, 48 weeks, 51 weeks, 54 weeks, 57 weeks, 60 weeks, 75 weeks, 90 weeks, and 120 weeks respectively.

Dosages for a therapeutic (e.g., docetaxel or cetuximab) in a combination therapy can, in some embodiments, be the same for each treatment cycle or the therapeutic's dosages may vary per cycle. In some embodiments, a higher initial dose of a therapeutic is administered for the first cycle and a lower dose is administered for all subsequent cycles. In other embodiments, a therapeutic's dosages are decreased gradually per administration for each cycle. In yet other embodiments, a therapeutic's dosages are increased gradually per administration for each cycle.

Administration of a wortmannin analog can, in some embodiments, be provided daily to a subject, i.e., a continuous dose, when receiving one of the described administration regimens for the other therapeutic (e.g., docetaxel or cetuximab) in a combination therapy. In other embodiments, wortmannin analogs are provided every other day, every 2 days, every 3 days, every 4 days, every 5 days, every 6 days or every 7 days to a subject.

Administration of a wortmannin analog can, in some embodiments, also be provided in reference to the treatment cycles of the other therapeutic (e.g., docetaxel or cetuximab). In some embodiments, a wortmannin analog is administered daily or every other day, every 2 days and the like for one treatment cycle for the other therapeutic and subsequently for the next treatment cycle, wortmannin analog administration of wortmannin analog is withheld or given a “drug holiday”. In other embodiments, a wortmannin analog is given to a subject every other treatment cycle, every two treatment cycles, every three treatment cycles, every four treatment cycles, or every five treatment cycles of the other therapeutic.

Dosages for wortmannin analogs can, in some embodiments, be the same for each treatment cycle or the dosages may vary per cycle. In some embodiments, a higher initial dose of a wortmannin analog is administered for the first cycle and a lower dose is administered for all subsequent cycles. In other embodiments, the wortmannin analog dosages are decreased gradually per administration for each cycle. In yet other embodiments, the wortmannin analog dosages are increased gradually per administration for each cycle.

Administration of a wortmannin analog can, in other embodiments, also be provided in an intermittent dosing schedule. Intermittent dosing schedules include administering a wortmannin analog for a number of days, withholding administration for a certain period of time, subsequently administering a wortmannin analog again with another subsequent withholding. In a non-limiting example, for a 28-day treatment cycle, a wortmannin analog can be administered for days 1-5 and 8-12. Other intermittent dosing schedules are contemplated that include administration of a wortmannin analog daily for one, two, three, four, five, six, seven, eight, nine or ten days, a withholding period of one, two, three, four, five, six, seven, eight, nine or ten days and an optional daily and withholding period similar or different from the previous administration within a treatment cycle.

In certain embodiments, a wortmannin analog is provided the same day as administration of the other therapeutic (e.g., docetaxel or cetuximab) in a combination therapy. In yet other embodiments, wortmannin analogs are provided the previous day of an administration of the other therapeutic in a combination therapy. In yet other embodiments, a wortmannin analog is provided the subsequent day of an administration of the other therapeutic. In certain instances where a wortmannin analog is provided prior to an administration of the other therapeutic, a wortmannin analog can be provided multiple days prior to administration of the other therapeutic, including, administration of a wortmannin analog for two days prior, three days prior, four days prior, five days prior, six days prior, or seven days prior to administration of the other therapeutic.

When a wortmannin analog is provided the same day as an administration of another therapeutic in a combination therapy (e.g., docetaxel or cetuximab), the wortmannin analog may be administered at a set time in reference to the administration of the other therapeutic. In some embodiments, a wortmannin analog is administered about 1 hour, about 2 hours, about 4 hours, about 6 hours, about 8 hours, about 12 hours, about 18 hours, about 24 hours prior to administration of the other therapeutic. In some embodiments, a wortmannin analog is administered about 1 hour, about 2 hours, about 4 hours, about 6 hours, about 8 hours, about 12 hours, about 18 hours, about 24 hours subsequent to administration of the other therapeutic.

In some embodiments when a wortmannin analog is administered orally, the oral administration is given to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, a wortmannin analog is administered orally to a subject who is in a fasted state for at least 8 hours. In other embodiments, a wortmannin analog is administered orally to a subject who is in a fasted state for at least 10 hours. In yet other embodiments, a wortmannin analog is administered orally to a subject who is in a fasted state for at least 12 hours. In other embodiments, a wortmannin analog is administered orally to a subject who has fasted overnight.

In other embodiments when a wortmannin analog is administered orally, the oral administration is given to a subject who is in a fed state. A fed state refers to a subject who has taken food or has had a meal. In certain embodiments, a wortmannin analog is administered orally to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, a wortmannin analog is administered orally to a subject in a fed state 30 minutes post-meal.

In other instances, a wortmannin analog is administered orally to a subject in a fed state 1 hour post-meal. In yet further embodiments, a wortmannin analog is administered orally to a subject with food.

In further embodiments of the wortmannin analog combinations described herein with another therapeutic (e.g., docetaxel or cetuximab), the wortmannin analog is administered at a certain time of day for the entire administration period. For example, a wortmannin analog can be administered at a certain time in the morning, in the evening, or prior to bed. In certain instances, a wortmannin analog is administered in the morning. In other embodiments, a wortmannin analog can be administered at different times of the day for the entire administration period. For example, a wortmannin analog can be administered in 8:00 am in the morning for the first day, 12 pm noon for the next day or administration, 4 pm in the afternoon for the third day or administration, and so on.

Any administration of the wortmannin analog combinations described herein with another therapeutic (e.g., docetaxel or cetuximab) can be adjusted and modified accordingly via factoring conditions as a subject's response, age, sex, disease, etc. at the beginning of treatment and throughout the course of the administration. Administration of a wortmannin analog and another therapeutic are also adjusted and modified according to a desired bioavailability of the two agents. In some embodiments, bioavailability of a wortmannin analog is measured by a wortmannin analog metabolite described herein in the subject such as the 17-hydroxy or 11,17-hydroxy metabolite.

Any administration of the combination therapies described herein can be further adjusted and modified accordingly via factoring conditions as a subject's response, age, sex, disease, etc at the beginning of treatment and throughout the course of the administration.

In any of the aforementioned embodiments, the wortmannin analog is PX-866, or salt, solvate, or polymorph thereof. In any of the aforementioned embodiments, the wortmannin analog is 17-hydroxy PX-866, or salt, solvate, or polymorph thereof.

### Pharmacokinetics of Wortmannin Analogs

In further embodiments of the combination therapies provided herein, a wortmannin analog (e.g., a compound of Formula IA, Formula IB, Formula IIA, Formula IIB, PX-866 or PX-867) is administered to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 250 pg/mL and about 5000 pg/mL (peak) within about 1-8 hours of administration of the wortmannin analog.

In another embodiment, the wortmannin analog (e.g., a compound of Formula IA, Formula IB, Formula IIA, Formula IIB, PX-866 or PX-867) is administered to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 500 pg/mL and about 4000 pg/mL (peak) within about 1-8 hours of administration of the wortmannin analog.

In another embodiment, the wortmannin analog (e.g., a compound of Formula IA, Formula IB, Formula IIA, Formula IIB, PX-866 or PX-867) is administered to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 500 pg/mL and about 2500 pg/mL (peak) within about 1-3 hours of administration of the wortmannin analog.

In another embodiment, the wortmannin analog (e.g., a compound of Formula IA, Formula IB, Formula IIA, Formula IIB, PX-866 or PX-867) is administered to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 600 pg/mL and about 2000 pg/mL (peak) within about 1-3 hours of administration of the wortmannin analog.

In yet another embodiment, the wortmannin analog (e.g., a compound of Formula IA, Formula IB, Formula IIA, Formula IIB, PX-866 or PX-867) is administered to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 750 pg/mL and about 1900 pg/mL (peak) within about 1-3 hours of administration of the wortmannin analog.

In yet another embodiment, the wortmannin analog (e.g., a compound of Formula IA, Formula IB, Formula IIA, Formula IIB, PX-866 or PX-867) is administered to an individual in need thereof at a dose and frequency of administration sufficient to result in a plasma concentration of the wortmannin analog (e.g., a compound of Formula IA, Formula IB, Formula IIA, Formula IIB, PX-866 or PX-867) and/or an active metabolite thereof (e.g., 17-hydroxy PX-866, 17-hydroxy PX-867) between about 750 pg/mL and about 1750 pg/mL (peak) within about 1-3 hours of administration of the wortmannin analog.

In some specific embodiments, for any of the aforementioned embodiments, the wortmannin analog is PX-866 and/or an active metabolite thereof (e.g., 17-hydroxy PX-866). In some specific embodiments, for any of the aforementioned embodiments, the wortmannin analog is a 17-hydroxy metabolite of PX-866.

In other embodiments of the combination therapies provided herein, a wortmannin analog (e.g., a compound of Formula IA, Formula IB, Formula IIA, Formula IIB, PX-866 or PX-867) is administered to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 250 pg/mL and about 5000 pg/mL.

In another embodiment, the wortmannin analog (e.g., a compound of Formula IA, Formula IB, Formula IIA, Formula IIB, PX-866 or PX-867) is administered to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 500 pg/mL and about 4000 pg/mL.

In another embodiment, the wortmannin analog (e.g., a compound of Formula IA, Formula IB, Formula IIA, Formula IIB, PX-866 or PX-867) is administered to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 600 pg/mL and about 3000 pg/mL.

In yet another embodiment, the wortmannin analog (e.g., a compound of Formula IA, Formula IB, Formula IIA, Formula IIB, PX-866 or PX-867) is administered to an individual in need thereof at a dose and frequency of administration sufficient to provide a plasma Cₘₐₓ of the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867) between about 750 pg/mL and about 2000 pg/mL.

In some specific embodiments, for any of the aforementioned embodiments, the wortmannin analog is PX-866 and/or an active metabolite thereof (e.g., 17-hydroxy PX-866). In some specific embodiments, for any of the aforementioned embodiments, the wortmannin analog is a 17-hydroxy metabolite of PX-866.

In yet other embodiments of the combination therapies provided herein, a wortmannin analog (e.g., a compound of Formula IA, Formula IB, Formula IIA, Formula IIB, PX-866 or PX-867) is administered to an individual in need thereof at a dose and frequency of administration sufficient to provide an AUC of between about 500 hr*pg/mL and about 12,000 hr*pg/mL for the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867).

In another embodiment, the wortmannin analog (e.g., a compound of Formula IA, Formula IB, Formula IIA, Formula IIB, PX-866 or PX-867) is administered to an individual in need thereof at a dose and frequency of administration sufficient to provide an AUC of between about 1000 hr*pg/mL and about 10,000 hr*pg/mL for the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867).

In yet another embodiment, the wortmannin analog (e.g., a compound of Formula IA, Formula IB, Formula IIA, Formula IIB, PX-866 or PX-867) is administered to an individual in need thereof at a dose and frequency of administration sufficient to provide an AUC of between about 2000 hr*pg/mL and about 8000 hr*pg/mL for the wortmannin analog (e.g., a compound of Formula IA, Formula IB, Formula IIA, Formula IIB, PX-866 or PX-867) and/or a metabolite thereof (e.g., 17-hydroxy PX-866, 17-hydroxy PX-867).

In some specific embodiments, for any of the aforementioned embodiments, the wortmannin analog is PX-866 and/or an active metabolite thereof (e.g., 17-hydroxy PX-866). In some specific embodiments, for any of the aforementioned embodiments, the wortmannin analog is a 17-hydroxy metabolite of PX-866.

In further embodiments of the combination therapies provided herein, a wortmannin analog (e.g., a compound of Formula IA, Formula IB, Formula IIA, Formula IIB, PX-866 or PX-867) is administered to an individual in need thereof at a dose and frequency of administration sufficient to reduce and/or alleviate incidence of proteinuria and/or elevated ALT/AST. In some specific embodiments, the wortmannin analog is PX-866 and/or an active metabolite thereof (e.g., 17-hydroxy PX-866). In some specific embodiments, the wortmannin analog is a 17-hydroxy metabolite of PX-866.

### Further Combinations

The combinations of a wortmannin analog with another therapeutic (e.g., docetaxel or cetuximab) described herein encompass additional therapies and treatment regimens with other agents in some embodiments. Such additional therapies and treatment regimens can include another anti-cancer therapy in some embodiments. Alternatively, in other embodiments, additional therapies and treatment regimens include other agents used to treat adjunct conditions associated with the cancer or a side effect from either a wortmannin analog or the other therapeutic in the combination therapy. In further embodiments, adjuvants or enhancers are administered with a combination therapy described herein.

Additional anti-cancer therapies include chemotherapy, radiotherapy, immunotherapy, gene therapy, surgery or other therapies that are capable of negatively affecting cancer in a patient, such as for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

Chemotherapies include, but are not limited to, hormone modulators, androgen receptor binding agents (e.g., anti-androgens, bicalutamide, flutamide, nilutamide, MDV3100), gonadotropin-releasing hormone agonists and antagonists (e.g., leuprolide, buserelin, histrelin, goserelin, deslorelin, nafarelin, abarelix, cetrorelix, ganirelix degarelix), androgen synthesis inhibitors (abiraterone, TOK-001), temozolomide, mitozolomide, dacarbazine, cisplatin (CDDP), carboplatin, procarbazine, mechlorethamine, cyclophosphamide, camptothecin, ifosfamide, melphalan, chlorambucil, busulfan, nitrosurea, dactinomycin, anthracyclines (e.g., daunorubicin, doxorubicin, epirubicin, idarubicin), bleomycin, plicomycin, mitomycin, etoposide (VP16), tamoxifen, raloxifene, estrogen receptor binding agents, cabazitaxel, paclitaxel, gemcitabine, navelbine, farnesyl-protein transferase inhibitors, transplatinum, 5-fluorouracil, capecitabine, vincristin, vinblastin and methotrexate, topoisomerase inhibitors (e.g., irinotecan, topotecan, camptothecin, etoposide) or any derivative related agent of the foregoing. Many of the above agents are also referred to as hormone therapy agents such as, for example, androgen receptor binding agents, gonadotropin-releasing hormone agonists and antagonists, androgen synthesis inhibitors, estrogen receptor binding agents as well as aromatase inhibitors. In other embodiments, the combination therapies provided herein are administered with irinotecan. In yet other embodiments, the combination therapies provided herein are administered with topotecan.

Radiotherapies include factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is likely that all of these factors affect a broad range of damage on DNA, on the precursors of DNA, on the replication and repair of DNA, and on the assembly and maintenance of chromosomes. Dosage ranges for X-rays may range from daily doses of 50 to 200 roentgens for prolonged periods of time (e.g., 3 to 4 weeks), to single doses of 2000 to 6000 roentgens. Dosage ranges for radioisotopes vary widely, and depend on the half-life of the isotope, the strength and type of radiation emitted, and the uptake by the neoplastic cells. In some embodiments, the combination therapies described herein are administered with a radiotherapy.

Immunotherapies, generally, rely on the use of immune effector cells and molecules to target and destroy cancer cells. The immune effector may be, for example, a tumor antigen or an antibody specific for some marker on the surface of a tumor cell. The tumor antigen or antibody alone may serve as an effector of therapy or it may recruit other cells to actually effect cell killing. An antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. Alternatively, an tumor antigen may stimulate a subject's immune system to target the specific tumor cells using cytotoxic T cells and NK cells. In some embodiments, the combination therapies described herein are administered with an immunotherapy. In other embodiments, the combination therapies described herein are administered with Sipuleucel-T (Provenge®). In other embodiments, the combination therapies described herein are administered with bevacizumab.

In other embodiments, an additional anti-cancer therapy is a gene therapy in which a therapeutic polynucleotide is administered before, after, or at the same time as a combination therapy. Therapeutic genes may include an antisense version of an inducer of cellular proliferation (oncogene), an inhibitor of cellular proliferation (tumor suppressor), or an inducer of programmed cell death (pro-apoptotic gene). In some embodiments, the combination therapies described herein are administered with a gene therapy.

In further embodiments, surgery of some type is performed in conjunction with the combination therapies described herein. Surgery types include preventative, diagnostic or staging, curative and palliative surgery and can be performed prior to, during, or subsequent to the combination therapy.

The mammalian target of rapamycin (mTOR) is a highly conserved intracellular serine/threonine kinase and a major downstream component in the PI3K pathway. Certain studies demonstrate that the PI3K-Akt-mTOR pathway mediates the response induced by EGFR activation. Accordingly, in some embodiments, combination therapies described herein further comprise administration of small molecule EGFR tyrosine kinase inhibitors (e.g., gefitinib, erlotinib or the like) in combination with wortmannin analogs and another therapeutic for prevention, delayed progression, reversal and/or partial reversal of established cancers and/or cancers that are refractory to other treatments. In some embodiments, the combination therapies described herein further comprise administration of wortmannin analogs in combination with small molecule mTor inhibitors including and not limited to rapamycin, temsirolimus, deforolimus, everolimus, BEZ235 or the like.

In some embodiments, an additional agent used to treat adjunct conditions associated with the cancer or a side effect from either the wortmannin analog or another therapeutic (e.g., docetaxel or cetuximab) in the combination therapy. Additional agents include, but are not limited to, anti-inflammatories, anti-emetics, anti-diarrheals and analgesics. In certain instances, the additional agents are administered prophylactically or as a pre-treatment prior to the wortmannin analog or other therapeutic. In other instances, the additional agents are administered on a needed basis, i.e., when a condition or side effect arises.

Anti-inflammatories can be used to treat or reduce the incidence and severity of, for example, inflammatory conditions, fluid retention or hypersensitivity reactions that result from the one or both of the agents in the combination therapy and/or conditions from the cancer. Anti-inflammatories are often given to patients with certain cancers such as glioblastoma to reduce peritumoral edema, diminish mass effect, lower intracranial pressure and reduce headache or drowsiness. Anti-inflammatories include, but are not limited to corticosteroids (e.g., dexamethasone, prednisone, hydrocortisone, betamethasone, triamcinolone and the like); NSAIDS such as arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam); and anti-histamines such as cimetidine, ranitidine, famotidine and nizatidine.

Anti-emetics can be used to treat nausea or vomiting associated with the cancer or one or both of the agents of the combination therapy. Anti-emetics include 5-HT receptor antagonists (ondansetron, granisetron, dolasetron, tropisetron, palonosetron, mirtazapine, etc.), dopamine antagonists (haloperidol, droperidol, prochlorperazine, etc.), antihistamines such as H₁ antagonists (promethazine, diphenhydramine, meclizine, etc.), benzodiazepines (lorazepam, midazolam), cannabinoids, and dexamethasone. Other known anti-emetics can be used as in conjuncation with the combination therapy in some embodiments.

Anti-diarrheals can be used to treat or prevent diarrhea associated with the cancer or one or both of the agents of the combination therapy. Anti-diarrheals include bismuth subsalicylate, loperamide, diphenoxylate, difenoxin, as well as other opioids.

Analgesics can be used to acute or chronic pain associated with the cancer or one or both of the agents of the combination therapy. Analgesics include acetaminophen, NSAIDS and opioid drugs (morphine, hydromorphone, fentanyl, tramadol, oxymorphone, oxycodone, hydrocodone, etc.) and COX-2 inhibitors.

In further embodiments; other additional agents for use with combination therapies described herein include immunosuppressants such as, for example, corticosteroids, gamma-interferon, Serum Amyloid P, azathioprine, penicillamine, cyclosporine, mycophenolate mofetil, or the like. Other additional therapeutic agents include colchicine, perfenidone or the like.

### Effects of Treatment

Treatment with combination therapies described herein may result in various effects. One effect of treating a subject, with a combination therapy described herein is an increase in the length of survival. Similarly, administering a described combination therapy to a subject may impact that subject's “quality of life” or “health-related quality of life.” Moreover, in certain subjects, treatment with a combination therapy described herein results in modulating assessed biomarkers including, but not limited to, decreases in phosphatase and tensin homolog (PTEN) mutational status, PI3K gene amplification, PI3K catalytic subunit alpha (PIK3CA) mutational status, K-ras mutational status, and/or B-raf mutational status as well as biomarkers specific in various cancers. For example, a treatment with a described combination therapy to an individual with prostate cancer can lower prostate-specific antigen (PSA), stabilize PSA, or decrease PSA doubling rates.

Comparisons of the effects of treatment with a combination therapy described herein can be made between treated subjects and subjects who are either undergoing no care, subjects who are undergoing a standard of care (SOC) or subjects who are receiving only one of the active agents in a combination therapy described herein. SOC comprises many alternative types of care that do not include treatment with a combination therapy described herein. For example, SOC, although usually discretionary depending on the circumstances, may include psychosocial support, analgesics, and nutritional support. In some embodiments, comparison of the effects of treatment will be made between subjects receiving differing amounts of active agents a combination therapy described herein. In yet further embodiments, individuals will undergo SOC in conjunction with treatment with a combination therapy described herein.

In some embodiments, before treatment of a subject with a combination therapy described herein, the subject may undergo pre-treatment evaluation. A non-limiting example of a pre-treatment evaluation includes a complete history and physical examination. The physical examination may include such things as a CT scan or X-ray. Subjects may also undergo treatment evaluations during the course of treatment. A treatment evaluation may include monitoring a subject's vital signs, inspecting injection sites, and analyzing blood samples.

In some embodiments, a treated subject with a with a described combination therapy, may have treatment effects evaluated by determining: (a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life. Tumor evaluations can be determined by any standardized criteria including Response Evaluation Criteria In Solid Tumors (RECIST) criteria.

Survival rates can be determined by comparing the current number of survivors with the number of individuals who started treatment a described combination therapy. In other embodiments, survival rates can be compared to published survival rates for a particular type of cancer. In yet other embodiments, survival rates can be compared to survival rates of individuals treated with one of the active agents in a combination therapy. In general, the survival rate may be measured at any time following the start of treatment.

For example, the survival rate may be measured at less than 6 months following the start of treatment, greater than 6 months but less than a year, a year or greater but less than 2 years, 2 years or greater but less than 5 years, or 5 or greater years. In some embodiments, an increased survival rate will be evidence that a described combination therapy has effects on a particular subject.

In some embodiments, an effect of treating a subject having cancer a combination therapy described herein is maintenance or an increase in a subject's quality of life. Clinicians and regulatory agencies recognize that a subject's “quality of life” (“QoL”) is an important endpoint in cancer clinical trials. See, for instance, Plunkett et al., Clin. Lung Cancer, 5(1):28-32 (2003), and Cella et al., J. Clin. Epidemiol., 55(3):285-95 (2002), which are each incorporated herein by reference.

Four important quality of life indicators are physical and occupational function, psychologic state, social interaction, and somatic sensations. For example, in individuals with NSCLC, two lung cancer questionnaires, the European Organization for Research and Treatment of Cancer (“EORTC”) and the Functional Assessment of Cancer Therapy (“FACT-L”), can be used to assess an individual's, specifically an individual's, health-related quality of life before, during, and after treatment with a combination therapy described herein.

In various embodiments, the above evaluations may be used in conjunction with assessments according to various subscales that monitor a subject's Physical Well-being (PWB), Social/Family Well-being (SWB), Emotional Well-being (EWB), Functional Well-being (FWB), and Lung Cancer Symptom subscale (LCS). Although the Lung Cancer Symptom subscale is obviously tailored to individuals with lung cancer, different subscales may be used with different types of cancer. Thus, a different subscale may be used with individuals with other cancers. Depending on which “Well-being” scores are combined, one may obtain a “FACT-L score” (the sum of all of the subscales) or a “Trial Outcome Score (TOI)” (the sum of the PWB, FWB, and LCS subscales). The TOI is a reliable indicator of meaningful change in quality of life. See, Cella et al., supra.

A subject may be assessed for their FACT-L and TOI scores before, during, and after treatment with a combination therapy described herein. For instance, the TOI score may be taken at baseline, i.e., pre-treatment, and then at various intervals after treatment has started, i.e., at 4 weeks, 8 weeks, 19 weeks, 31 weeks, or 43 weeks, or longer. These various intervals are examples only and the quality of life indicators may be taken at any appropriate time. For example, the first TOI score may be taken after the first treatment, instead of at a baseline. Then, the change in scores between various time points may be calculated to determine trends relating to improving, worsening, or maintaining of quality of life.

It has been calculated that a decrease of 3 points or more from baseline for LCS is a clinically meaningful worsening in lung cancer symptoms and an increase in 3 or more points is a clinically meaningful improvement in lung cancer symptoms. Likewise for TOI scores, a decrease of 7 or more points indicates a worsening in quality of life, while an increase of 7 or more points indicates an improvement in quality of life.

In some embodiments, a clinical improvement in cancer symptoms or quality of life demonstrates that a described combination therapy has effects on a particular subject.

Administering a combination therapy described herein may be useful in improving or maintaining the quality of life of treated subjects that have cancer. In measuring the effect on the quality of life, an effect size can be determined from baseline or from any treatment point. In some embodiments, an effect size of between 0.2 to <0.49 indicates a small effect, 0.5 to 0.79 indicates a moderate effect, and 0.8 or greater indicates a large effect. These numbers are examples only and the effect size may change with treatment of certain subjects.

Administration of a combination therapy described herein may also be useful in preventing the worsening in quality of life seen over time in many cancer patients. For example, in some embodiments, administration of a combination therapy described herein may result in quality of life indexes that essentially remain unchanged or do not reach the level of worsening or improving quality of life.

In other embodiments, the present treatments described herein encompasses improving or maintaining the quality of life or improving or stabilizing lung cancer symptoms in an individual diagnosed with NSCLC by determining the individual's TOI or LCS scores before, during, and after treatment with a combination therapy described herein.

In other embodiments, the response of subjects to a combination therapy described herein is measured by changes in certain biomarkers including, but not limited, decreases in phosphatase and tensin homolog (PTEN) mutational status, PI3K gene amplification, PI3K catalytic subunit alpha (PIK3CA) mutational status, K-ras mutational status, and/or B-raf mutational status. Biomarkers include other changes in copy number, nucleotide and protein concentrations, and/or mutational status in other genes involved in one of the PI-3K signal transduction pathways. The effects of a combination therapy on biomarkers can be measured at any time. For example, although a PTEN copy number can be compared to a baseline value, PTEN copy number may also be compared between treatment points or between a specific treatment point and the end of treatment.

In further embodiments, the response of subjects to a combination therapy described herein is measured using tests of immune function, such as a T-cell proliferation response assay. In some embodiments, the results from T-cell proliferation response assays will be used to determine whether a combination therapy described herein has an effect on a subject. Results from these assays may also be used to determine individual response to the formulations during different time points during the course of the treatment. Comparison of the T-cell proliferation response may be undertaken to compare pre-treatment versus post-treatment response as well as to compare immune responses within treatment.

### Kits/Articles of Manufacture

For use in the combination therapies described herein, kits and articles of manufacture are also described herein. Such kits can comprise a carrier, package, or container that is compartmentalized to receive one or more containers such as vials, tubes, and the like, each of the container(s) comprising one of the separate elements to be used in a method described herein including a wortmannin analog and another therapeutic (e.g., docetaxel or cetuximab). Suitable containers include, for example, bottles, vials, syringes, and test tubes. The containers can be formed from a variety of materials such as glass or plastic.

A kit will typically may comprise one or more additional containers, each with one or more of various materials (such as reagents, optionally in concentrated form, and/or devices) desirable from a commercial and user standpoint for a combination therapy described herein. Non-limiting examples of such materials include, but not limited to, buffers, diluents, filters, needles, syringes; carrier, package, container, vial and/or tube labels listing contents and/or instructions for use, and package inserts with instructions for use associated with a wortmannin analog and/or another therapeutic (e.g., docetaxel or cetuximab). A set of instructions will also typically be included.

A label can be on or associated with the container. A label can be on a container when letters, numbers or other characters forming the label are attached, molded or etched into the container itself; a label can be associated with a container when it is present within a receptacle or carrier that also holds the container, e.g., as a package insert. A label can be used to indicate that the contents are to be used for a specific therapeutic application. The label can also indicate directions for use of the contents, such as in the methods described herein.

Kits can be supplied and manufactured according to dosages or administration methods described herein. For example, a kit can be supplied with a container for a 10 treatment cycle of docetaxel along with a 30 week supply of a wortmannin analog. In another example, a kit can be supplied with a container for a 5 treatment cycle of cetuxamib along with a 15 week supply of a wortmannin analog.

### DEFINITIONS

Unless defined otherwise, all technical and scientific terms used herein have the same meanings as commonly understood by one of ordinary skill in the art. Although any methods and materials similar or equivalent to those described herein can be used in the practice or testing of embodiments described herein, certain preferred methods, devices, and materials are now described.

As used herein and in the appended claims, the singular forms “a”, “an”, and “the” include plural reference unless the context clearly dictates otherwise. Thus, for example, reference to “a cell” is a reference to one or more cells and equivalents thereof known to those skilled in the art, and so forth.

The term “about” is used to indicate that a value includes the standard level of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

“Optional” or “optionally” may be taken to mean that the subsequently described structure, event or circumstance may or may not occur, and that the description includes instances where the events occurs and instances where it does not.

“Administering” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering”, when used in conjunction with a wortmannin analog or metabolite thereof, can include, but is not limited to, providing a wortmannin analog or metabolite thereof into or onto the target tissue; providing a wortmannin analog or metabolite thereof systemically to a patient by, e.g., intravenous injection whereby the therapeutic reaches the target tissue or cells.

“Administering” a composition may be accomplished by injection, topical administration, and oral administration or by other methods alone or in combination with other known techniques.

As used herein, the term “therapeutic” means an agent utilized to treat, combat, ameliorate, prevent or improve an unwanted condition or disease of a patient. In some embodiments, a therapeutic agent is directed to the treatment and/or the amelioration of, reversal of, or stabilization of the symptoms of a cancer described herein. Exemplary therapeutics include docetaxel and cetuxamib.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. Other examples of subjects include experimental animals such as mice, rats, dogs, cats, goats, sheep, pigs, and cows. The experimental animal can be an animal model for a disorder, e.g., a transgenic mouse with a glioblastoma pathology. A patient can be a human suffering from glioblastoma and variants or etiological forms.

The term “irreversible inhibitor” refers to an inhibitor that forms a covalent bond with the target moiety, in this case, P1-3 kinase.

By “pharmaceutically acceptable”, it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology). As such, a non-limiting example of a “therapeutically effective amount” or “effective amount” of a composition of the present disclosure may be used to inhibit, block, or reverse the activation, migration, or proliferation of cells or to effectively treat cancer or ameliorate the symptoms of cancer.

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

As used herein, “continuous dosing” means administration of at least one dose of a compound (e.g., a wortmannin analog and/or metabolite thereof) daily for a period of at least 7 days. In some embodiments, continuous dosing means administration of at least one dose of a compound (e.g., a wortmannin analog and/or metabolite thereof) daily for 1 week. In some embodiments, continuous dosing means administration of at least one dose of a compound (e.g., a wortmannin analog or and/or metabolite thereof) daily for 2 weeks. In some embodiments, continuous dosing means administration of at least one dose of a compound (e.g., a wortmannin analog and/or metabolite thereof) daily for 3 weeks. In some embodiments, continuous dosing means administration of at least one dose of a compound (e.g., a wortmannin analog and/or metabolite thereof) daily for 4 weeks. In some embodiments, continuous dosing means administration of at least one dose of a compound (e.g., a wortmannin analog and/or metabolite thereof) daily for 5 or more weeks.

Optionally, in some embodiments, continuous dosing alternates with a drug holiday in a cyclical treatment regimen. Accordingly, by way of example, in some embodiments, continuous dosing means administration of a first cycle of at least one dose of a compound (e.g., a wortmannin analog and/or metabolite thereof) daily for a period of at least one week followed by a drug holiday of up to two weeks, followed by administration of one or more further cycles of administration of at least one dose of a compound (e.g., a wortmannin analog and/or metabolite thereof) daily for a period of at least one week followed by a drug holiday of up to two weeks. In some embodiments, continuous dosing means administration of a first cycle of at least one dose of a compound (e.g., a wortmannin analog and/or metabolite thereof) daily for a period of at least 2 weeks followed by a drug holiday of up to two weeks, followed by administration of one or more further cycles of administration of at least one dose of a compound (e.g., a wortmannin analog and/or metabolite thereof) daily for a period of at least 2 weeks followed by a drug holiday of up to 2 weeks. In some embodiments, continuous dosing means administration of a first cycle of at least one dose of a compound (e.g., a wortmannin analog and/or metabolite thereof) daily for a period of at least 3 weeks followed by a drug holiday of up to two weeks, followed by administration of one or more further cycles of administration of at least one dose of a compound (e.g., a wortmannin analog inhibitor and/or metabolite thereof) daily for a period of at least 3 weeks followed by a drug holiday of up to 2 weeks. In some embodiments, continuous dosing means administration of a first cycle of at least one dose of a compound (e.g., a wortmannin analog inhibitor and/or metabolite thereof) daily for a period of at least 4 weeks followed by a drug holiday of up to two weeks, followed by administration of one or more further cycles of administration of at least one dose of a compound (e.g., a wortmannin analog and/or metabolite thereof) daily for a period of at least 4 weeks followed by a drug holiday of up to 2 weeks. In some embodiments, continuous dosing means administration of a first cycle of at least one dose of a compound (e.g., a wortmannin analog and/or metabolite thereof) daily for a period of 5 or more weeks followed by a drug holiday of up to two weeks, followed by administration of one or more further cycles of administration of at least one dose of a compound (e.g., a wortmannin analog and/or metabolite thereof) daily for a period of 5 or more weeks followed by a drug holiday of up to 2 weeks.

In some embodiments of a continuous dosing regimen, the drug holiday between two cycles of dosing is about 2 weeks. In some embodiments of a continuous dosing regimen, the drug holiday between two cycles of dosing is about 10 days. In some embodiments of a continuous dosing regimen, the drug holiday between two cycles of dosing is about 1 week. In some embodiments of a continuous dosing regimen, the drug holiday between two cycles of dosing is about 5 days. In some embodiments of a continuous dosing regimen, the drug holiday between two cycles of dosing is about 3 days.

As used herein, “intermittent dosing” means administration of a first cycle of at least one dose of a compound (e.g., a wortmannin analog and/or metabolite thereof) daily for a period of between about 2 to about 5 days, followed by a drug-free period of between about 2 to about 25 days, followed by one or more such cycles.

The term “wortmannin analog” or “analog of wortmannin” refers to any compounds in which one or more atoms, functional groups, or substructures in wortmannin have been replaced with different atoms, groups, or substructures while retaining or improving upon the functional activity of wortmannin and/or improving PK profiles and/or reducing toxicity of wortmannin.

### EXAMPLES

### Example 1

### A Phase I Trial of Oral PX-866 in Patients with Advanced Solid Tumors

This was an Open-label dose escalation study with expansion cohort at Maximal Tolerated Dose (MTD), 3+3 design and to test efficacy of 2 dosing schedules (intermittent and continuous). PX-866 was administered as an oral dose.

### Study Objectives

The primary and secondary objectives were tested using two different dosing regimens: 10 days of drug administration and daily administration for 28 days.

### Primary:

To determine the MTD of PX-866 when administered to patients with advanced metastatic cancers.
To evaluate the safety profile of PX-866 when administered orally on a 28 day schedule.
To evaluate pharmacodynamic measures of the effects of PX-866 on the phosphatidylinositol-3 kinase (PI-3K) pathway and related tumor markers.
To determine the PK profile of PX-866 when adminstered orally on a 28 day schedule.

### Secondary:

To evaluate the anti-tumor activity of PX-866 in patients with advanced malignancies.

### Selected Eligibility Criteria

Inclusion Criteria:
≧18 years at time of consent
Able to give an informed consent
Has a histologically or cytologically confirmed diagnosis of advanced solid tumor and has failed or is intolerant of standard therapy, or for whom standard therapy does not exist
Eastern Cooperative Oncology Group (ECOG) performance of 0 or 1
Life expectancy of at least 12 weeks
Discontinued prior chemotherapy or other investigational agents for at least three weeks prior to receiving the first dose of study drug (six weeks for mitomycin C, nitrosureas, vaccines, or antibody therapy) and recovered from the toxic effects of the prior treatment (recovered to baseline or grade 1 per Common Toxicity Criteria for Adverse Events
Discontinued any radiation therapy for at least four weeks and have recovered from all radiation-related toxicities (recovered to baseline or CTCAE grade 1) prior to receiving the first dose of study drug. Palliative radiation of 10 fractions or less is permitted and a four week interval is not necessary (also allowed during therapy).
Laboratory requirements:

| WBC count | >3,000 | cells/μL; |
|---|---|---|
| Platelets | >100,000/μL | |
| Hemoglobin | >9 | g/dL |
| ANC | >1,500 | cells/μL |
| Bilirubin | >1.5 | mg/dL |
| Aminotransferases (ALT and AST) | <2.5 × ULN or <5 × ULN due | |
| to metastatic disease | | |
| Serum Creatinine | <1.5 | mg/dL |

Women of childbearing potential agree to use adequate contraception (hormonal or barrier method; abstinence) prior to study entry and for the duration of study participation.

Exclusion Criteria:
Any active infection
Known diabetes or fasting blood glucose>160 mg/dL
Known HIV
Any serious concomitant systemic disorders that in the opinion of the investigator would place the patient at excessive or unacceptable risk of toxicity
Surgery within the four weeks prior to the first dose of PX-866
Significant central nervous system (CNS) or psychiatric disorder(s) that preclude the ability of the patient to provide informed consent
Known or suspected brain metastases that have not received adequate therapy
Patients with a history of seizures, non-healing wounds, or arterial thrombosis
Patients with unstable atrial or ventricular arrhythmias requiring control by medication
Patients who are breastfeeding or pregnant
Patients with total gastrectomy, partial bowel obstruction or any gastrointestinal condition that may interfere with absorption of the study medication
Any condition that could jeopardize the safety of the patient and compliance with the protocol

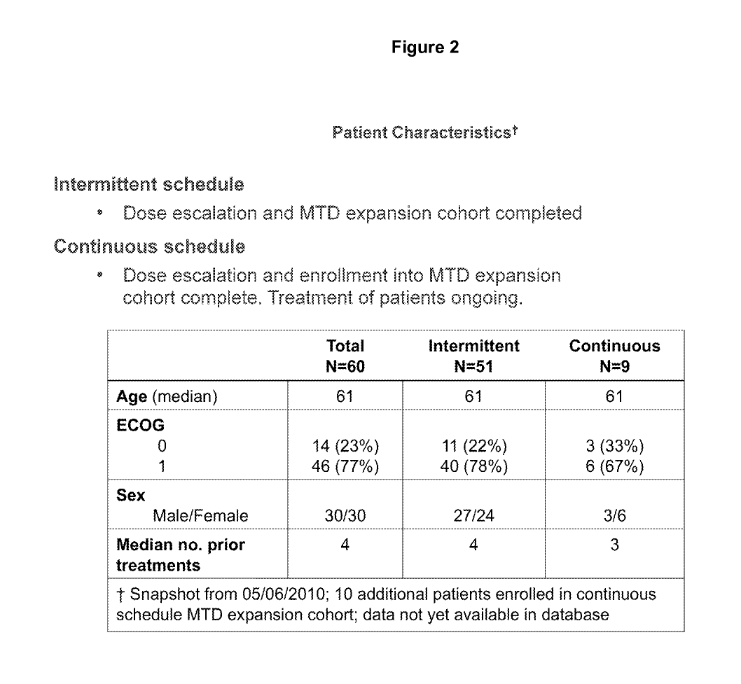

FIG. 2 describes a breakdown of patient characteristic from a May 6, 2010 snapshot.

Safety Levels with Intermittent Dosing

Intermittent schedule: 10 dose levels tested (0.5-16 mg). The starting dose level was 0.5 mg. Doses increased as follows: 100% escalation up to 2 mg, 50% escalation up to 4.5 mg, and approximately 30% escalation until the MTD is identified. The highest dose level at which no more than ⅙ patients experiences DLT was declared the MTD. The resulting dose levels were: 0.5, 1, 2, 3, 4.5, 6, 8, 10, 12 and 16 mg.

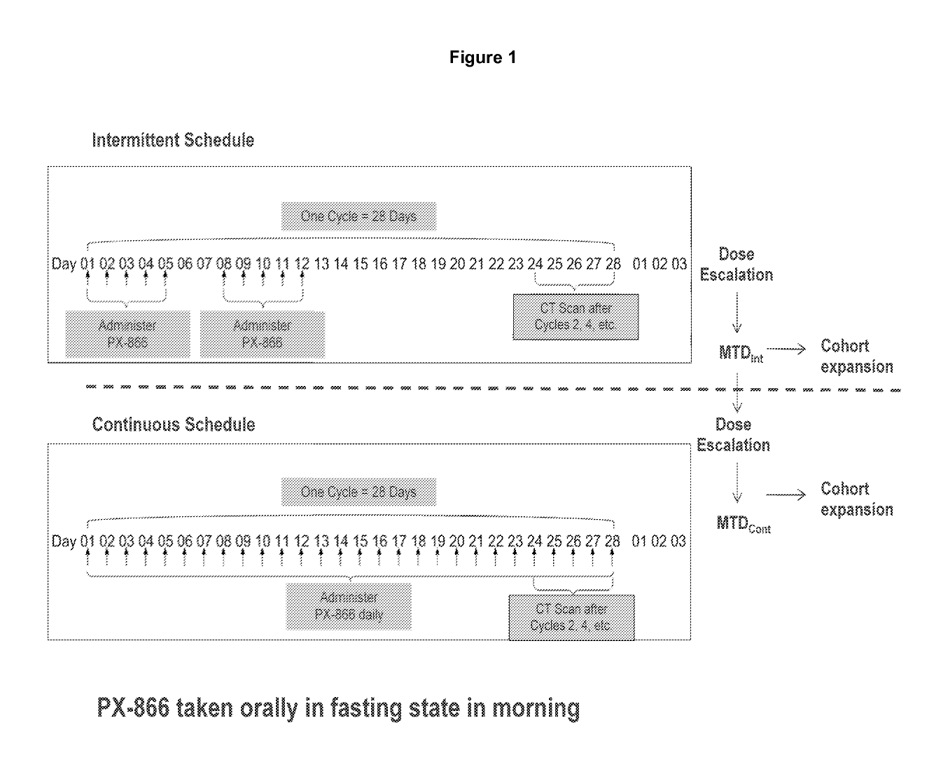

FIG. 1 illustrates the dosing schedule for intermittent dosing where PX-866 was given to patients on days 1-5 and 8-12 of a 28-day cycle.

At a dose of 16 mg per day, Dose limiting toxicity (DLT) was observed in 2/5 patients treated at 16 mg. Grade 3 diarrhea (n=1); Grade 3 AST (n=1) were observed.

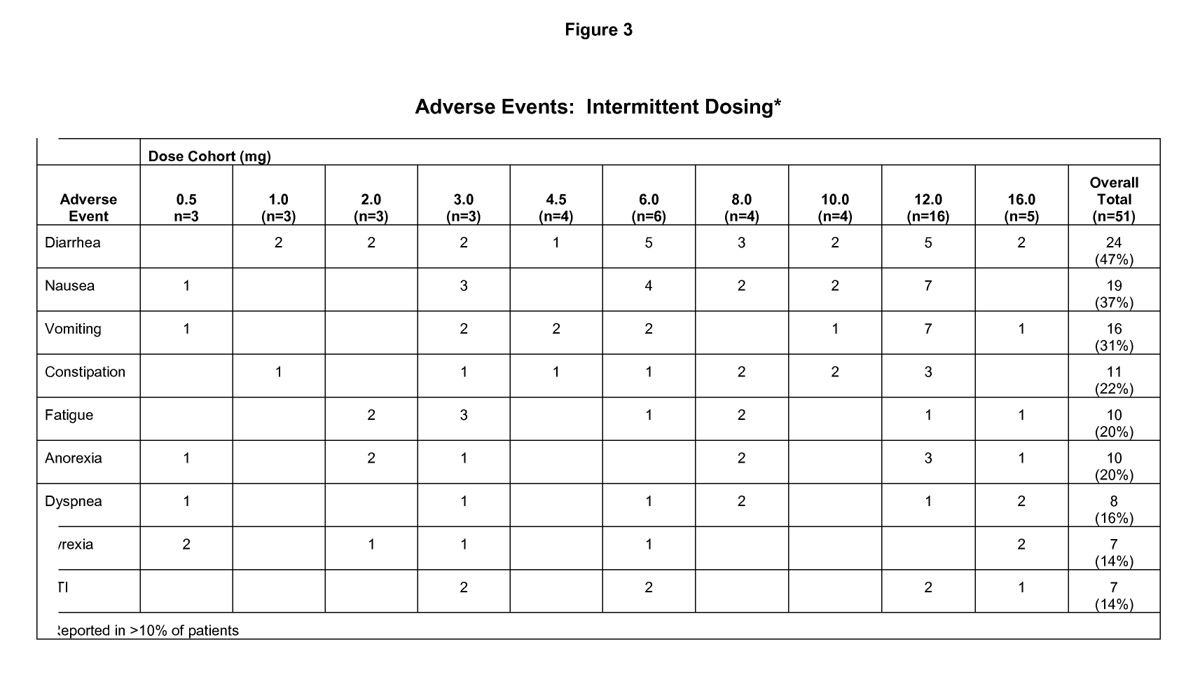

Most common adverse events (AEs) included diarrhea, nausea, vomiting, and constipation. FIG. 3 describes adverse events with intermittent dosing. Related Grade 3 events included vomiting (n=3), diarrhea (n=3), liver enzyme elevation (n=2) dehydration (n=1), and worsened hypertension (n=1).

No significant increase in toxicity was observed in patients receiving>2 cycles with intermittent dosing schedule.

The Maximal Tolerated Dose (MTD) for intermittent dosing was determined as 12 mg per day.

Safety Levels with Continuous Dosing

Continuous dosing schedule: The starting dose was two dose levels below the MTD of intermittent dosing (8 mg) and subsequent dose levels was one or two dose levels (10 mg or 8 mg) below the MTD of intermittent dosing dependent on recommendation of the dose cohort review committee. FIG. 1 illustrates the dosing schedule for continuous dosing.

At a dose of 10 mg per day, DLT was observed in 2/3 patients treated at 10 mg. Grade 3 diarrhea (n=2) was observed.

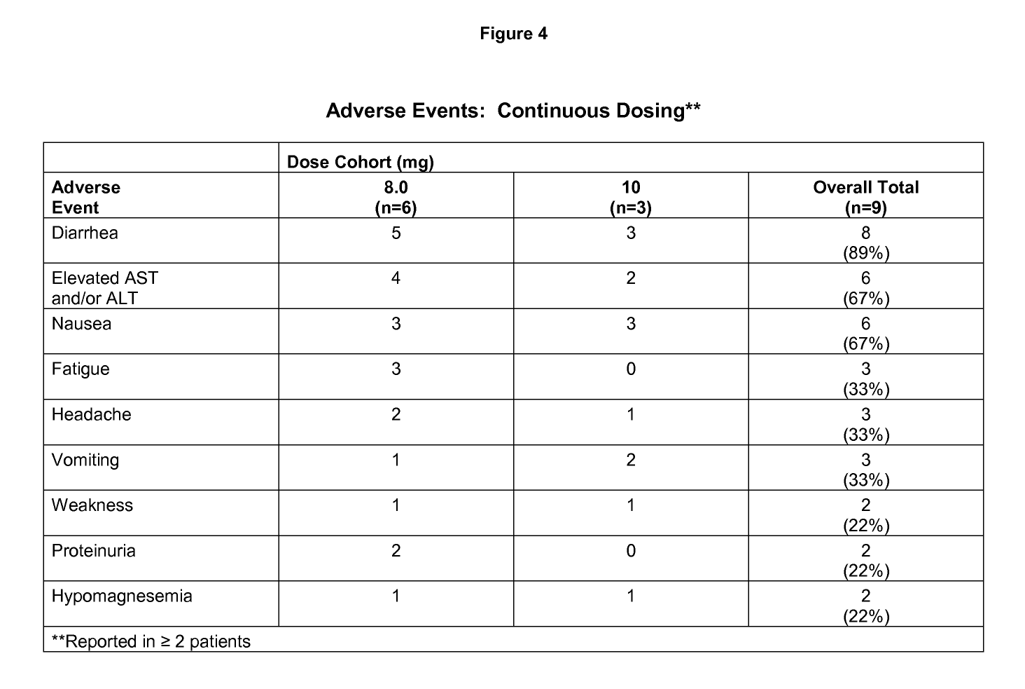

Most common adverse events (AEs) include diarrhea, nausea, vomiting, headache and fatigue. ALT/AST elevation was observed with continuous dosing. Related Grade 3 events include vomiting (n=3), diarrhea (n=3), liver enzyme elevation (n=2) dehydration (n=1), worsened hypertension (n=1). FIG. 4 describes adverse events with continuous dosing.

No significant increase in toxicity was observed in patients receiving>2 cycles with intermittent dosing schedule.

The Maximal Tolerated Dose (MTD) for continuous dosing was determined as 8 mg per day.

### Study Response

Patient response was evaluated according to Response Evaluation Criteria in Solid Tumors (RECIST). Briefly, all measurable lesions up to a maximum of five lesions, representative of all involved organs were identified as target lesions and recorded and measured at baseline. Target lesions were selected on the basis of their size (lesions with the longest diameter) and their suitability for accurate repeated measurements (either by imaging techniques or clinically). A sum of the longest diameter (LD) for all target lesions was calculated and reported as the baseline sum LD. The baseline sum LD was used as reference by which to characterize the objective tumor. All other lesions (or sites of disease) were identified as non-target lesions and were also be recorded at baseline. Measurements of these lesions were not required, but the presence or absence of each was noted throughout follow-up.

A Complete Response (CR) indicated a disappearance of all target lesions. A Partial Response (PR) showed at least a 30% decrease in the sum of the LD of target lesions, taking as reference the baseline sum LD. Progressive Disease (PD) was defined as at least a 20% increase in the sum of the LD of target lesions, taking as reference the smallest sum LD recorded since the treatment started or the appearance of one or more new lesions and Stable Disease (SD) indicated that there was neither sufficient shrinkage to qualify for PR nor sufficient increase to qualify for PD, taking as reference the smallest sum LD since the treatment started.

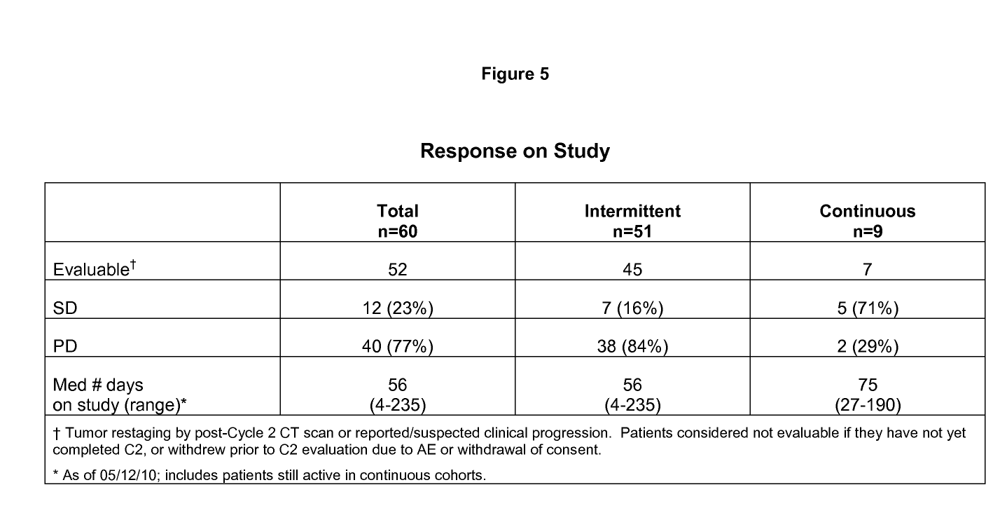

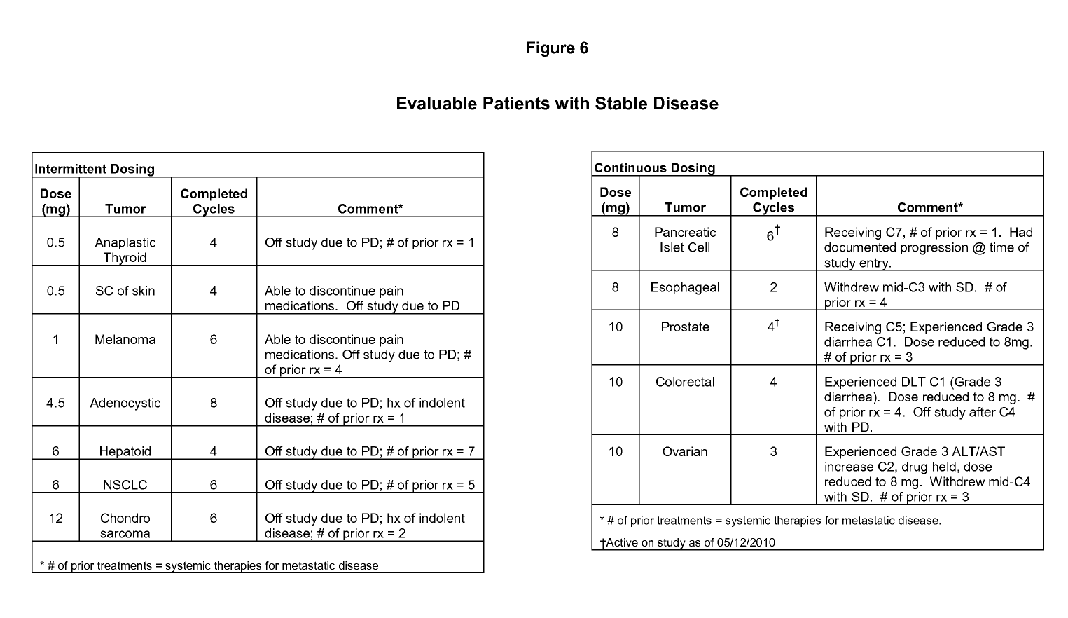

FIG. 5 describes response to intermittent and continuous dosing studies. Disease stabilization was observed in 71% of patients undergoing continuous dosing. Disease stabilization was observed in 16% of patients undergoing intermittent dosing. Patients, who were prior treated with other drugs but had subsequent disease progression, benefited from treatment with PX-866. PX-866 treatment stabilized disease in prior treated patients. FIG. 6 describes evaluable patients with stable disease that had prior treatments. The number of prior treatments with other drugs ranged from 1 to 7 for these patients, with a median of 4 prior systemic therapeutic regimens for metastatic disease.

### Clinical Pharmacokinetics

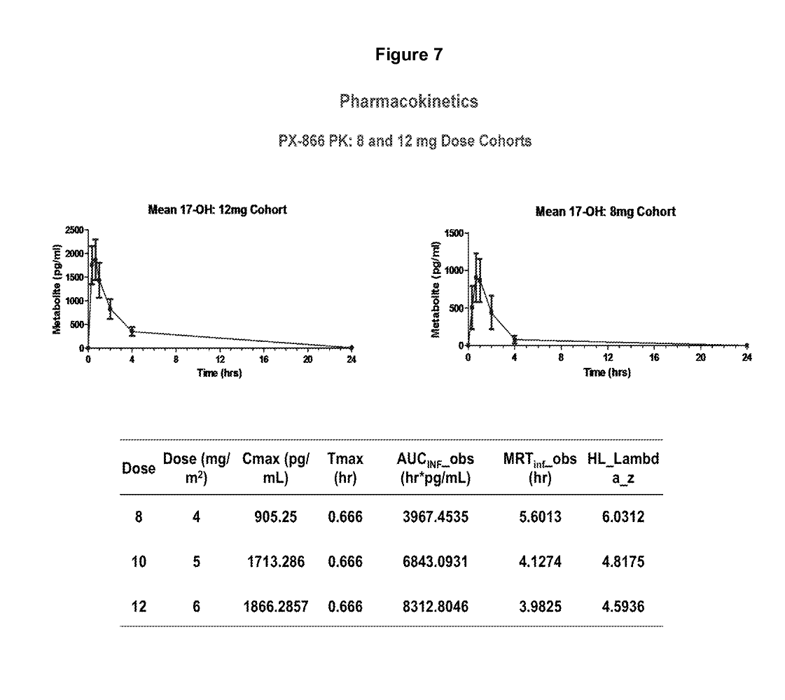

Pharmacokinetic studies revealed evidence of rapid conversion of PX-866 to a 17-OH metabolite. With rare exceptions, the parent PX-866 was below the limits of detection. Production of the 17-OH metabolite was rapid, with a Tₘₐₓ ranging from 0.67-1.07 hours. FIG. 7 depicts the pharmacokinetics of PX-866 administration in humans in 8 and 12 mg cohorts for intermittent dosing. Analysis of the 12 mg cohort revealed that the pharmacokinetics of the 17-OH metabolite showed no evidence of drug accumulation.

Analysis of data from patients dosed at the continuous dosing of 8 mg showed a mean Cₘₐₓ of 1140 pg/mL, an AUC₍₀₋₂₄₎ of 4220 hr*pg/mL and a half-life of 3.62 hr.

It was particularly noted that the Cmax of the 17-OH metabolite was equal to or exceed peak levels observed in mice treated at an efficacious dose of PX-866 (2 mg/kg). In addition the AUC for the 17-OH metabolite in humans exceeded AUC in mice due to an increase in mean residence time in humans.

### Clinical Pharmacodynamics

The pharmacodynamic effects of PX-866 in patients treated in the phase I single agent study were assessed using isolated peripheral blood mononuclear cells (PBMCs) stimulated ex vivo via FACS based assay. PX-866 treatment was associated with inhibition of the PI-3K pathway as assessed by changes in the downstream kinases p-mTOR and p-S6. The study provided Evidence for pathway inhibition lasting up to 3 days post-treatment.

Additional pharmacodynamic data from patients on the phase I study of PX-866 indicate that 3 of 4 patients treated at the 8 mg dose level of PX-866 had a 60% or greater decrease in p-AKT/T-AKT 4 hours after a single oral dose of drug.

### Example 2

### Effect of PX-866 and Docetaxel in a Lung Tumor Xenograft Animal Model

PX-866 and docetaxel was tested in a lung tumor xenograft animal model to evaluate the effects of the combination on survival of the animals. H460 and A549 lung tumor xenografts were implanted subcutaneously in nude mice according to previously described procedures. PX-866 was tested on both a daily (2 mg/kg) and every-other-day dosing (3 mg/kg) schedule alone, and in combination with a 10 mg/kg dose of docetaxel. The dosing schedule of PX-866 relative to docetaxel was varied to test the effect of docetaxel treatment prior to or subsequent to PX-866 treatment. A table of the following dosing regimens is as follows with animals in Group 1 receiving a daily vehicle:

| | Dosing for H460 & A549 Models | |
|---|---|---|
| Group | PX-866 | Docetaxel |
| Group 1 | — | — |
| Group 2 | 2 mg/kg daily | — |
| Group 3 | 3 mg/kg every-other-day | — |
| Group 4 | — | 10 mg/kg weekly |
| Group 5 | 2 mg/kg daily; 6 hours prior to docetaxel | 10 mg/kg weekly |
| Group 6 | 3 mg/kg every-other-day; 6 hours prior to | 10 mg/kg weekly |
| | docetaxel | |
| Group 7 | 2 mg/kg daily; 24 hours after docetaxel | 10 mg/kg weekly |
| Group 8 | 3 mg/kg every-other-day; 24 hours after | 10 mg/kg weekly |
| | docetaxel | |

In the H460 model, the animals that received combination therapy with PX-866 and docetaxel survived significantly longer than animals treated with docetaxel alone, with the exception of animals treated with the combination of PX-866 at 3 mg/kg administered 24 hours after 10 mg/kg docetaxel. In general, greater decreases in body weight were observed in animals treated with combination therapy compared to animals treated with single agent therapy.

The results of the A549 Lung Tumor xenograft groups showed PX-866 treatment alone, and in combination with docetaxel, resulted in greater tumor growth delay relative to the control group. Increased mortality was observed in the 2 mg/kg PX-866 daily treatment groups, as well as the docetaxel combination groups.

### Example 3

### Effect of PX-866 and Docetaxel in a Direct Patient Tumor Model of Squamous Cell Carcinoma of the Head and Neck

PX-866 and docetaxel was tested in a direct patient tumor model (DPTM) of squamous sell carcinoma of the head and neck (SCCHN) to evaluate the effects of the combination on tumor growth. A direct patient tumor model was developed to preserve key features of human disease, including better replication of tumor-stroma interactions and preservation of human cancer stem cells. Briefly, these models employed direct implantation of patients' tumors into nude mice according to Keysar et al., J Clin Oncol 28:15s, 2010 (suppl. abstruct 5558).

PX-866 and docetaxel were evaluated in a DPTM for CUHN015 tumors using a 2 mg/kg every day for 5 out of 7 days schedule. Animals were treated with PX-866 alone, and in combination with 20 mg/kg docetaxel dosed intraperitoneally (IP) on a weekly schedule. Saline treated animals and 20 mg/kg docetaxel dosed animals served as controls for the study. Animals bearing the CUHN015 tumors, derived from a human papilloma virus negative laryngeal squamous cell carcinoma, treated with PX 866 alone showed a delay in tumor growth and the combination of docetaxel and PX 866 resulted in tumor stasis. Additionally, docetaxel alone had no effect on tumor growth in the CUHN015 model suggesting that the combination of PX-866 with docetaxel was synergistic in this model.

### Example 4

### Phase ½ Open-label Study of PX-866 and Docetaxel Given to Patients with Locally Advanced, Recurrent, or Metastatic Cancer

### Study Objectives

### Phase 1

Primary: To determine the maximum tolerated dose (MTD) or recommended dose (RD) of PX-866 to be administered orally once per day in combination with docetaxel administered IV at a dose of 75 mg/m² once every 21 days in patients with incurable locally advanced, recurrent, or metastatic cancer.

Secondary: To evaluate the preliminary antitumor activity of PX-866 administered in combination with docetaxel as assessed by objective response rate (ORR) and early progression rate (% of patients with progressive disease at 6 weeks).

### Phase 2

Primary: To evaluate the antitumor effects of PX-866 administered at the MTD/RD in combination with docetaxel as assessed by objective response rate (ORR) and early progression rate in up to 3 disease indications including incurable locally advanced, recurrent, or metastatic non-small cell lung cancer (NSCLC), incurable locally advanced, recurrent, or metastatic squamous cell carcinoma of the head and neck (SCCHN), and up to 1 other disease indication to be determined.

### Phase 1 and Phase 2

Secondary:
To evaluate the safety and tolerability as assessed by the incidence and severity of adverse events and laboratory abnormalities of PX-866 administered at the MTD/RD orally once per day in combination with docetaxel
To characterize the pharmacokinetics of docetaxel when administered in combination with PX-866
To characterize the pharmacokinetics of PX-866 when administered in combination with docetaxel
To evaluate the antitumor effects of PX-866 administered at the MTD/RD in combination with docetaxel as assessed by the disease control rate (DCR; the proportion of patients with complete remission [CR], partial remission [PR] or stable disease [SD])

Exploratory: To evaluate potential predictive biomarkers of PX-866 and docetaxel activity

### Study Population

Phase 1: Patients with incurable locally advanced, recurrent, or metastatic cancer for which docetaxel administered at a dose of 75 mg/m² IV every 21 days is approved, is considered standard of care (SOC), or has been compendia listed.

Phase 2: Up to 3 different disease-specific populations is evaluated in 3 separate cohorts including:

1) patients with NSCLC who have received at least 1 and no more than 2 prior systemic treatment regimens for incurable locally advanced, recurrent, or metastatic disease that may include up to 1 platinum-based chemotherapy regimen and/or an epidermal growth factor receptor (EGFR) inhibitor and
2) patients with SCCHN who have received no more than 2 prior systemic treatment regimens for incurable locally advanced, recurrent, or metastatic disease that may include up to 1 platinum-based chemotherapy regimen and/or an EGFR inhibitor.
If activity is observed in Phase 1 patients for a different indication, a third cohort of patients with this indication may be enrolled in Phase 2.

Inclusion Criteria:
≧18 years at time of consent
Signed an informed consent
Consent to using a medically accepted form of contraception from the time of consent to completion of all follow-up study visits, if sexually active
Measurable disease per Response Evaluation Criteria In Solid Tumors (RECIST) or if castrate-resistant prostate cancer (CRPC) patient, evaluable for response or progression based on prostate-specific antigen (PSA) or changes in bone scan
Documentation available for last prior systemic treatment including dates of treatment, best response to treatment, duration of best response, and reason for discontinuation of treatment
Eastern Cooperative Oncology Group (ECOG) 0 or 1
In the opinion of the clinical investigator, life expectancy>3 months
Adequate hematologic function as defined by:
   Hemoglobin≧9 g/dL
   Absolute neutrophil count (ANC)≧1500 cells/4
   Platelets≧100,000/4
Adequate hepatic function as defined by the following:
   Bilirubin ≦ULN (unless documented history of Gilbert's disease)
   Aspartate aminotransaminase (AST/SGOT) and alanine aminotransferase (ALT/SGPT)≦1.5× upper limit of normal (ULN)
   Alkaline phosphatase≦2.5 ULN
Creatinine level≦1.5×ULN

Exclusion Criteria:
Medical, social, or psychosocial factors that, in the opinion of the investigator, could impact safety or compliance with study procedures
Pregnant or Breastfeeding
Treatment with any systemic chemotherapy, epidermal growth factor receptor (EGFR) inhibitor, radiation or experimental agent within 4 weeks of study drug dosing, or treatment with an antiandrogen within 6 weeks of study drug dosing
Previous treatment with docetaxel except for patients with CRPC in Phase 1. Patients with CRPC in Phase 1 may have received up to one prior treatment regimen containing docetaxel as adjuvant therapy or as treatment for metastatic disease
Previous treatment with a phosphatidylinositol 3-kinase (PI-3K) inhibitor
Known human immunodeficiency virus (HIV)
Known or suspected clinically active brain metastases. Previously treated and stable brain metastases are allowable
Grade>2 peripheral neuropathy, as defined by the National Cancer Institute (NCI) Common Terminology Criteria for Adverse Events (CTCAE), Version 4.02
Any other significant medical or psychiatric condition that in the opinion of the investigator renders the patient inadequate for participation
History of severe hypersensitivity reactions to docetaxel or to other drugs formulated with polysorbate

### Study Design

This is a Phase ½ open-label study of PX-866 given in combination with docetaxel to patients with incurable locally advanced, recurrent, or metastatic cancer (Phase 1) or patients receiving treatment for locally advanced, recurrent, or metastatic NSCLC, SCCHN, or third yet to be determined indication to determine efficacy potential (Phase 2).

### Phase 1

Phase 1 of this study evaluates up to 3 dose levels of PX-866 to determine the MTD or RD of PX-866 to be given orally once per day in combination with docetaxel 75 mg/m² administered IV once every 21 days. The MTD is evaluated in cohorts of 6 evaluable patients.

Once the MTD/RD is determined, additional patients with solid tumors is enrolled and treated at that dose level in a safety expansion cohort (for a total of at least 15 evaluable patients treated at the MTD/RD) to further evaluate the safety, tolerability, and pharmacokinetics of the combination. If at any time 33% or more of the patients treated at the MTD/RD experience dose-limiting toxicity (DLT), enrollment is halted and the study Safety Monitoring Committee (SMC) will review all available safety data to determine if enrollment may continue at that dose level, or if a lower dose should be evaluated prior to proceeding to Phase 2.

To allow for assessment of the effects of PX-866 on docetaxel pharmacokinetics, treatment in Phase 1 consists of docetaxel administered on Day 1 of Cycle 1 followed by initiation of treatment with daily oral PX 866 on Day 8. For all subsequent cycles, docetaxel and PX-866 is administered on Day 1 of the treatment cycle.

### Dose Escalation

After establishing eligibility, patients are enrolled in the current dose cohort of PX-866 administered in combination with docetaxel. Doses of PX-866 that are studied are provided in the table below. The starting dose of PX-866 is 50% of the single agent MTD of 8 mg (i.e., 50% of 8 mg=4 mg).

PX-866 Dose Cohorts

| Cohort | PX-866 Dose |
|---|---|
| Cohort 1 | 4 mg |
| Cohort 2 | 6 mg |
| Cohort 3 | 8 mg |

The SMC can recommend additional dose levels to be evaluated, including those that are less than 8 mg.

### Dose Escalation Guidelines

Dose escalation for PX-866 is based on safety data from the first 2 treatment cycles from cohorts of 6 evaluable subjects. The SMC reviews patient data prior to escalating to the next planned dose cohort. For the purpose of this SMC review, a patient is considered evaluable if they have received at least 75% of the planned doses of PX-866 during Cycle 1 (≧11/14 doses) and Cycle 2 (≧16/21 doses). If the reason for not receiving 75% or more of the planned doses is DLT or other PX-866 related toxicity, a patient will still be considered evaluable for this SMC review. Patients considered non-evaluable are replaced.

Once a cohort is open, up to 3 patients are enrolled. Once at least 2 evaluable patients successfully complete therapy through Day 21 without experiencing DLT, then the remainder of patients in the cohort are enrolled. The following dose-escalation guidelines are followed for each dose cohort:
If no more than 1 patient of the first 6 evaluable patients treated experience DLT, then the dose of PX-866 will be escalated
If 2 or more patients within the first 6 evaluable patients treated experience DLT, then this dose level will be considered not tolerated

Patient accrual into any dose cohort is stopped as soon as 2 patients treated experience DLT. The study SMC subsequently reviews all available safety and PK data to determine if this dose should be declared not tolerated and if no further dose escalation should occur.

Once dose escalation has been halted, the SMC convenes to declare a MTD or RD for further study in the expanded safety cohort. The SMC also reviews safety data from subjects receiving additional treatment cycles of PX-866 in combination with docetaxel; however, the decision to dose escalate is formally based on safety data from the first 2 treatment cycles.

### Definitions of Dose-Limiting Toxicities

Dose-limiting toxicities (DLT5s), defined using the National Cancer Institute (NCI) Common Terminology Criteria for Adverse Events (CTCAE) Version 4.02 (Oct. 26, 2009), are events that occur following administration of PX-866 and docetaxel combination therapy during Cycles 1 or 2, and meet the criteria described below. Events for which there is an alternative clinical explanation (e.g., clearly related to an intercurrent illness or disease progression) is not considered DLT. It is at the discretion of the investigator to determine if a toxicity that is considered exclusively or typically related to docetaxel can also be excluded from DLT definition.

### DLT Criteria

Any clinical adverse event≧Grade 3 in severity possibly, probably, or definitely related to study drug (PX-866+docetaxel) treatment with the exception of nausea, vomiting or diarrhea without maximal anti-emetic or anti-diarrheal therapy. Grade 3 or 4 alopecia will not be considered a DLT.
Any clinical adverse event≧Grade 3 in severity possibly, probably, or definitely related to study drug (PX-866+docetaxel) treatment with the exception of nausea, vomiting or diarrhea without maximal anti-emetic or anti-diarrheal therapy. Grade 3 or 4 alopecia will not be considered a DLT.

### Hematologic

Absolute neutrophil count (ANC) of Grade 3 or 4 plus fever (fever must be present for the Grade 3 or 4 ANC to be considered a DLT, and is defined as a temperature of 38.5° C. or greater)
ANC of <500/μL for >7 days
Platelet count of <25,000/4

### Hepatic

Grade 3 elevation of transaminases (ALT or AST) if persistent for >7 days
Grade 4 elevation of transaminases (ALT or AST)

### Endocrine

Grade 3 or greater increase in serum glucose if persistent despite optimal therapy

The relationship of adverse events to study drug treatment is determined by the investigator. Patients who experience a DLT can continue in the study. Such patients may resume treatment at the dose level below that at which the DLT occurred after recovery of the toxicity to no more than Grade 1 or the baseline level of severity.

### Phase 2

Phase 2 of the study evaluates the antitumor activity (as evaluated by ORR and early progression rate) and safety of PX-866 administered at the MTD/RD in combination with docetaxel in up to 3 disease-specific cohorts of patients. The disease-specific indications include patients receiving 2^{nd} or 3^{rd} line treatment for incurable locally advanced, recurrent, or metastatic NSCLC or patients receiving 2′¹ or 3^{rd} line treatment for incurable locally advanced, recurrent, or metastatic SCCHN. The study SMC makes a final recommendation prior to the start of Phase 2 regarding whether both indications will be studied and whether a third indication should also be evaluated.

### Test Product, Dose, and Mode of Administration

All treatments are administered on a 21-day cycle. Docetaxel 75 mg/m² is administered IV on Day 1 of each 21-day cycle. For Phase 1 only, PX-866 is administered orally once per day on Days 8 to 21 of Cycle 1 and Days 1 to 21 of all subsequent cycles. For Phase 2, PX-866 is administered orally once per day on Days 1 to 21 of all treatment cycles. Any patient with CRPC who is enrolled in the study receives prednisone 5 mg twice daily on Days 1 to 21 of each treatment cycle. Patients with CRPC receive dexamethasone 8 mg orally at 12, 3 and 1 hour prior to docetaxel administration. All patients other than CRPC patients are administered dexamethasone 8 mg orally twice daily for 3 days starting 1 day before administration of docetaxel.

### Number of Planned Patients

A maximum of 126 evaluable patients are enrolled in this study. Phase 1 enrolls a maximum of 36 evaluable patients, including up to 27 evaluable patients in 3 cohorts during dose escalation and approximately 9 patients in a safety expansion cohort at the MTD/RD. A maximum of 45 evaluable patients is initially enrolled in Phase 2 of the study, including 15 evaluable patients in up to 3 disease specific cohorts. Based upon the observed number of responses and the number of patients with early progression in a given cohort, an additional 15 evaluable patients may be enrolled in that cohort.

### Duration of Treatment

Patients are evaluated for progression approximately every 6 weeks. Patients with stable disease (SD) or better per investigator assessment receive repeat cycles of PX-866 and docetaxel on a 21-day schedule until disease progression, unacceptable toxicity, or withdrawal of consent.

### Efficacy Assessments

For patients with measurable disease, efficacy is evaluated per RECIST. Patients with CRPC can also be evaluated for PSA response per the Prostate Cancer Clinical Trials Working Group (PCWG2) recommendations.

### Safety Assessments

Safety assessments include surveillance and documentation of adverse events, laboratory assessments, and physical exam findings.

### Pharmacokinetic Assessments

Pharmacokinetic (PK) assessments include measurement of plasma levels of PX-866 and metabolites as well as plasma levels of docetaxel.

### Biomarker Assessments

Exploratory biomarker assessments include evaluation of the tumor mutational profile, including but not limited to phosphatase and tensin homolog (PTEN) mutational status, PI3K gene amplification, PI3K catalytic subunit alpha (PIK3CA) mutational status, K-ras mutational status, and B-raf mutational status.

### Endpoints

### Phase 1

Primary Endpoint: Incidence and severity of adverse events

Secondary Endpoint: Objective response rate (ORR) and early progression rate (% of patients with progressive disease at 6 weeks)

### Phase 2

Primary Endpoint: Objective response rate (ORR) and early progression rate (% of patients with progressive disease at 6 weeks)

Secondary Endpoint: Incidence and severity of adverse events and overall survival

### Phase 1 and Phase 2

### Secondary Endpoints

Incidence and severity of clinical laboratory abnormalities
Duration of response
Disease control rate (DCR; the proportion of patients with CR, PR or SD) at 6, 12, 18 and 24 weeks
Progression-free survival (PFS)
Plasma concentrations of PX-866 and metabolites
Plasma concentrations of docetaxel
Frequency of dose reductions in docetaxel
Frequency of dose reductions in PX-866

### Exploratory Endpoints

Tumor mutational profile, including but not limited to phosphatase and tensin homolog (PTEN) mutational status, PI3K gene amplification, PI3K catalytic subunit alpha (PIK3CA) mutational status, K-ras mutational status, and B-raf mutational status

### Statistical Methods

Phase 1: Phase 1 of the study enrolls a maximum of 36 evaluable patients, including a maximum of 37 evaluable patients in 3 cohorts during dose escalation and approximately 9 evaluable patients in an expanded safety cohort treated at MTD/RD (for a minimum of 15 evaluable patients to be treated at the MTD/RD). The sample size allows for an approximately 33% early discontinuation rate due to non-PX-866 related events. A dose is considered not tolerated if the observed rate of DLT in 15 patients was 33% (95% confidence interval 15-58%). With a sample size of 15 patients, if the true incidence of DLT is 10%, there is a 79% probability of observing at least 1 DLT and a 45% probability of observing 2 or more DLTs.

Phase 2: A maximum of 45 evaluable patients are enrolled initially in Phase 2, including 15 evaluable patients each in up to 3 disease-specific cohorts. A sample size of 15 is based on the first stage of a multinomial screening Phase 2 design utilizing response and early progression rates as described by Zee et al. to determine if a treatment warrants further evaluation. The combination of PX-866 and docetaxel is considered of interest for further study if the true response rate is 20% or greater AND if the true early progression rate is 40% or lower; the drug combination is be considered as warranting further study if the response rate is 5% or less AND the early progression rate is 60% or more. Based on these proportions, the combination of PX-866 and docetaxel is not considered of interest for further study if within the first 15 evaluable patients treated in a given cohort, there are:
0 responses are observed OR
No more than 1 response AND 7 or more early progressions are observed OR
No more than 3 responses AND 9 or more early progressions are observed.

If these criteria are not met, then the SMC may recommend enrollment of 15 additional patients within the given cohort.

### Evaluation of Safety

Adverse events are coded according to the Medical Dictionary for Regulatory Activities (MedDRA). Safety data are summarized by dose cohort and presented by individual treatment course as well as pooled over treatment courses. The severity of clinical adverse events and laboratory data are graded using NCI CTCAE, Version 4.02.

### Evaluation of Efficacy

Efficacy is evaluated according to RECIST and where applicable, per the Prostate Cancer Clinical Trials Working Group (PCWG2) recommendations. Independent radiological review is performed on computed tomography (CT) or magnetic resonance imaging (MRI) scans from patients in Phase 2 only if criteria are met for further study of the combination of PX-866 and docetaxel.

### Evaluation of Pharmacokinetic Activity

Non-compartmental pharmacokinetic modeling is used to estimate PK parameters for PX-866 and metabolites, as well as for docetaxel.

### Safety Monitoring Committee (SMC)

During the dose-escalation phase of the study, the SMC will meet to review adverse events and laboratory toxicities for the current dose cohort to determine if escalation to the next sequential dose cohort is warranted. During Phase 2 of the study, the SMC will perform ongoing reviews of safety, PK, and efficacy data on a 4 to 8 week interval and ad hoc basis. The SMC will also recommend whether criteria are met to move to the second stage of accrual.

### Example 5

### Effect of PX-866 and Cetuximab in a Pancreatic Tumor Xenograft Animal Model

PX-866 and cetuximab was tested in a lung tumor xenograft animal model to evaluate the effects of mean tumor volume in the animals. Bx-PC3 pancreatic tumor xenografts were implanted subcutaneously in nude mice similar to procedures previously described in Ihle et al, Mol Canc Ther, 2005 4(9): 1349-57. PX-866 was tested using both i.v. (10 mg/kg) as well as oral (3 mg/kg) dose routes in combination with 0.35 mg cetuximab using an every 2-day schedule when tumor volume was about 100 mm³ in the animals.

Animals were divided into six groups that received the following treatments: Group 1 received vehicle control (i.v.); Group 2 received PX-866 (i.v. 10 mg/kg) alone; Group 3 received PX-866 (oral 3 mg/kg) alone; Group 4 received cetuximab (i.p. 0.35 mg) alone, Group 5 received PX-866 (i.v. 10 mg/kg)+cetuximab (i.p. 0.35 mg); and Group 6 received PX-866 (oral 3 mg/kg)+cetuximab (i.p. 0.35 mg).

The combination of PX-866 with cetuximab in both dose route cohorts resulted in improved tumor control relative to cetuximab or PX-866 alone. In the groups that received both cetuximab and PX-866, mean tumor volume remained about 100 to 200 mm³ after 50 days post-implantation whereas mean tumor volume in all other groups was observed to begreater than 200 mm³ in the equivalent time frame.

### Example 6

### Effect of PX-866 and Cetuximab in a Direct Patient Tumor Model of Squamous Cell Carcinoma of the Head and Neck

PX-866 and cetuximab was tested in two direct patient tumor models (DPTM) of squamous sell carcinoma of the head and neck (SCCHN) to evaluate the effects of the combination on tumor growth. A direct patient tumor model was developed to preserve key features of human disease, including better replication of tumor-stroma interactions and preservation of human cancer stem cells. Briefly, these models employed direct implantation of patients' tumors into nude mice according to Keysar et al., J Clin Oncol 28:15s, 2010 (suppl. abstruct 5558).

PX-866 and cetuximab were evaluated in CUHN0022 and CUHN0027 SCCHN tumors. Animals were treated for four weeks with PX-866 at 2 mg/kg every day for 5 out of 7 days schedule alone or in combination with cetuximab dosed at 2 mg/kg on a twice-weekly schedule and observed. Saline treated animals and cetuximab dosed animals served as controls for the study.

Animals bearing CUHN022 tumors treated with cetuximab and PX-866 resulted in improved tumor control with a tumor growth inhibition of one-half as compared with saline treated animals. The tumor growth inhibition by the combination of cetuximab and PX-866 was also observed to be greater than animals treated with cetuximab or PX-866 alone.

Similarly, in the CUHN027 DPTM, the combination of PX-866 and cetuximab also resulted in improved tumor control relative to single agent cetuximab or PX-866 treatment. The differential effects of combination drug treatment on tumor growth were seen even after discontinuation of treatment.

### Example 7

### Phase ½ Open-label Study of PX-866 and Cetuximab Given to Patients with Locally Advanced, Recurrent, or Metastatic Cancer

### Study Objectives

### Phase 1

Primary: To determine the maximum tolerated dose (MTD) or recommended Phase 2 dose (RD) of PX-866 to be administered orally once per day in combination with cetuximab in patients with incurable metastatic colorectal carcinoma (CRC) or incurable progressive, recurrent or metastatic squamous cell carcinoma of the head and neck (SCCHN).

Secondary:
To evaluate the safety and tolerability of PX-866 administered at the MTD/RD in combination with cetuximab
To evaluate the preliminary antitumor activity of PX-866 administered in combination with cetuximab
To evaluate the effects of PX-866 on the pharmacokinetics (PK) of cetuximab

### Phase 2

Primary: To evaluate the antitumor effects of PX-866 administered in combination with cetuximab versus cetuximab alone in patients with incurable metastatic colorectal cancer (CRC) and/or in patients with incurable progressive, recurrent or metastatic squamous cell carcinoma of the head and neck (SCCHN).

Secondary:
To evaluate the safety and tolerability of PX-866 in combination with cetuximab versus cetuximab alone in patients with incurable metastatic CRC and/or in patients with incurable progressive, recurrent or metastatic SCCHN
To evaluate the effects of PX-866 administered at the MTD/RD on cetuximab PK
To evaluate the PK of PX-866 administered at the MTD/RD

### Phase 1 and Phase 2

Exploratory:
To evaluate potential pharmacodynamic markers of PX-866 and cetuximab activity
To evaluate potential predictive biomarkers of PX-866 and cetuximab activity

### Study Population

Eligible patients are those with incurable metastatic CRC or incurable progressive, recurrent or metastatic SCCHN who meet all of the following inclusion criteria:

Inclusion Criteria:
≧18 years at time of consent
Signed an informed consent
Consent to using a medically accepted form of contraception from the time of consent to completion of all follow-up study visits, if sexually active
Measurable disease per Response Evaluation Criteria In Solid Tumors (RECIST)
Documentation available for last prior systemic treatment including dates of treatment, best response to treatment, duration of best response, and reason for discontinuation of treatment
History of progression or recurrence following prior irinotecan and oxaliplatin containing regimens for metastatic disease or intolerance of irinotecan based therapy (Phase 2 CRC patients only)
Eastern Cooperative Oncology Group (ECOG) 0 or 1
In the opinion of the clinical investigator, life expectancy>3 months
Adequate hematologic function as defined by:
   Hemoglobin≧9 g/dL
   Absolute neutrophil count (ANC)≧1500 cells/μL
   Platelets≧100,000/μL
Adequate hepatic function as defined by the following:
   Bilirubin≦ULN (unless documented history of Gilbert's disease)
   Aspartate aminotransaminase (AST/SGOT) and alanine aminotransferase (ALT/SGPT)≦1.5× upper limit of normal (ULN)
Creatinine level≦1.5×ULN
Serum magnesium≧lower limit of normal (LLN).

Exclusion Criteria:
Medical, social, or psychosocial factors that, in the opinion of the investigator, could impact safety or compliance with study procedures
Pregnant or Breastfeeding
Treatment with any systemic chemotherapy, epidermal growth factor receptor (EGFR) inhibitor, radiation or experimental agent within 4 weeks of study drug dosing
Previous treatment with cetuximab (Phase 2 patients only; prior cetuximab is allowed for Phase 1 patients)
Previous treatment with a phosphatidylinositol 3-kinase (PI-3K) inhibitor
Known human immunodeficiency virus (HIV)
Poorly controlled diabetes mellitus (IFCC-HbA_{IC}≧53 mmol/mol or DCCT-HbA_{1C}≧7%)
Kras mutation in codon 12 or 13 (CRC patients only)
Known or suspected clinically active brain metastases. Previously treated and stable brain metastases are allowable
Any other significant medical or psychiatric condition that in the opinion of the investigator renders the patient inadequate for participation
History of severe hypersensitivity reactions to cetuximab

### Study Design

This is a Phase ½ open-label study. In the Phase 1 part of the study, PX-866 is given in combination with cetuximab to patients with incurable metastatic CRC or incurable progressive, recurrent or metastatic SCCHN. The Phase 2 part of the study is a randomized evaluation of the antitumor activity and safety of PX-866 in combination with cetuximab versus cetuximab alone in patients with either incurable metastatic CRC who have a history of progression or recurrence following prior irinotecan and oxaliplatin containing regimens or are intolerant of irinotecan (Group 1) or incurable progressive, recurrent or metastatic SCCHN (Group 2).

### Phase 1

Phase 1 determines the MTD or RD of PX-866 to be given orally on Days 1-21 in combination with cetuximab 250 mg/m² administered IV weekly on Days 1, 8, and 15 of a 21-day cycle. All patients receive an initial loading dose of 400 mg/m² cetuximab rather than 250 mg/m² on Cycle 1 Day 1. Patients may receive premedication with an H1 antagonist per the cetuximab package insert. Up to 3 dose levels of PX-866 are evaluated to determine the MTD/RD in cohorts of up to 6 patients using a standard 3+3 dose-escalation design. At least 6 patients are treated at the MTD/RD. All patients in Phase 1 are required to undergo PK assessments during Cycle 1 Week 3 to measure cetuximab levels. Exploratory PD assessments include evaluation of changes in levels of fasting C-peptide as well as changes in EGFR and PI-3K signaling pathways in peripheral blood mononuclear cells (PBMC) and platelets. Additional optional evaluations include changes in EGFR and PI-3K signaling in paired tumor biopsies provided before and after one cycle of treatment. All patients are asked, but not required, to provide an archived tumor biopsy sample for evaluation for potential biomarkers of response to PX-866 and cetuximab.

### Dose Escalation

After establishing eligibility, patients are enrolled in the current dose cohort of PX-866 administered in combination with cetuximab. The starting dose of PX-866 is 75% of the single agent MTD of 8 mg/day (i.e., 75% of 8 mg=6 mg). If 6 mg/day is well tolerated, the dose is escalated to 8 mg/day. If 6 mg/day is not tolerated, the dose is decreased to 4 mg/day. If 4 mg/day is not tolerated, an additional dose level of 2 mg/day may be evaluated.

### Dose Escalation Guidelines

Dose escalation for PX-866 are based on safety data from the first treatment cycle from cohorts of up to 6 evaluable patients. The study Safety Monitoring Committee (SMC) will review patient data prior to escalating to the next planned dose cohort. For the purpose of this SMC review, a patient is considered evaluable if they have received at least 14/21 of the planned doses of PX-866 during Cycle 1 and at least 2 of the 3 planned doses of cetuximab, as long as the reason for not receiving PX-866 or cetuximab was dose-limiting toxicity (DLT) or other treatment-related toxicity. Patients considered non-evaluable are replaced.

The first patient in each cohort is enrolled and followed through Day 8 of Cycle 1 prior to enrolling 2 additional patients. The following dose-escalation guidelines are followed for each dose cohort:
If no patient of the first 3 patients treated experience DLT, then the dose of PX-866 is escalated
If 1 patient of the first 3 patients treated experience DLT, then 3 additional patients is treated at that dose level
   If no more than 1 of the 6 patients experience DLT, then the dose of PX-866 is escalated
   If 2 or more patients within a cohort experience DLT, then this dose level is considered not tolerated and a lower dose level will be evaluated

Patient accrual into any dose cohort is stopped as soon as 2 patients treated experience DLT. The study SMC will review all available safety data to determine if this dose should be declared not tolerated and if no further dose escalation should occur.

Once dose escalation has been halted, the SMC convenes to declare an MTD or RD for further study in Phase 2. A minimum of 6 evaluable patients need to be treated at a dose level in order for that dose to be declared the MTD or RD. If the first 3 patients treated at a dose level do not experience DLT, and that is the dose level being considered for MTD/RD, then 3 additional patients are to be enrolled. The SMC also reviews safety data from patients receiving additional treatment cycles of PX-866 in combination with cetuximab; however, the decision to dose escalate is formally based on safety data from the first treatment cycle.

### Definitions of Dose-Limiting Toxicities

Dose-limiting toxicities (DLT5s), defined using the National Cancer Institute (NCI) Common Terminology Criteria for Adverse Events (CTCAE) Version 4.02 (Oct. 26, 2009), are events that occur following administration of PX-866 and cetuximab combination therapy during Cycle 1, and meet the criteria described below. Events for which there is an alternative clinical explanation (e.g., clearly related to an intercurrent illness or disease progression) will not be considered DLT. It will be at the discretion of the investigator to determine if a toxicity that is considered exclusively or typically related to cetuximab can also be excluded from DLT definition.

### DLT Criteria

Any clinical adverse event≧Grade 3 in severity possibly, probably, or definitely related to study drug (PX-866+cetuximab) treatment with the exception of nausea, vomiting or diarrhea without maximal anti-emetic or anti-diarrheal therapy, and Grade 3 papulopustular rash.
Any patient who requires greater than a 2-week delay in the start of Cycle 2 due to unresolved toxicity related to PX-866.

### Hematologic

Absolute neutrophil count (ANC) of Grade 3 or 4 plus fever (fever must be present for the Grade 3 or 4 ANC to be considered a DLT, and is defined as one reading of temperature>38.5° C., or 3 readings of temperature>38.0° C. in a 24-hour period)
ANC of <500/μL for >7 days
Platelet count of <25,000/μL

### Hepatic

Grade 3 elevation of transaminases (ALT or AST) if persistent for >7 days
Grade 4 elevation of transaminases (ALT or AST)

### Endocrine

Grade 3 or greater increase in serum glucose if persistent despite optimal therapy

The relationship of adverse events to study drug treatment is determined by the investigator. Patients who experience a DLT may continue in the study. Such patients may resume treatment at the dose level below that at which the DLT occurred after recovery of the toxicity to no more than Grade 1 or the baseline level of severity. Infusion reactions or hypersensitivity reactions to cetuximab is not considered DLT.

### Phase 2

Phase 2 of the study is an open-label, randomized evaluation of the antitumor activity and safety of PX-866 administered at the MTD/RD in combination with cetuximab, versus cetuximab alone in cetuximab-naïve patients with incurable metastatic CRC who have a history of progression or recurrence following prior irinotecan and oxaliplatin containing regimens or are intolerant of irinotecan (Group 1) or patients with incurable progressive, recurrent or metastatic SCCHN (Group 2). Seventy two evaluable patients (36 patients per arm) is evaluated per indication. Patients are randomized 1:1 to receive PX-866+cetuximab or cetuximab alone. Patients are considered evaluable for Phase 2 if they complete 2 treatment cycles and undergo post-cycle 2 tumor restaging unless the reason for not doing so is progressive disease or treatment-related toxicity. Patients who are randomized to receive cetuximab alone and develop progressive disease per RECIST may cross-over to receive PX-866 at the time of progression based upon the investigator's clinical assessment following discussion with the study medical monitor. The effect of PX-866 administered at the MTD/RD on cetuximab peak and trough levels at the RD is evaluated, as are levels of PX-866 and metabolites. All patients are asked, but not required, to provide an archived tumor biopsy sample for evaluation of potential biomarkers of response to PX-866 and cetuximab.

### Test Product, Dose, and Mode of Administration

All treatments are administered on a 21-day cycle. Cetuximab is administered IV once per week on Days 1, 8 and 15 of each treatment cycle. All patients receive an initial loading dose of 400 mg/m² cetuximab on Cycle 1 Day 1. All subsequent doses of cetuximab are 250 mg/m². Patients may receive pretreatment with an H1 antagonist prior to cetuximab administration per the cetuximab package insert. PX-866 is administered orally once per day on Days 1 to 21 of each treatment cycle in Phase 1 and at the MTD/RD for all patients randomized to combination treatment in Phase 2.

### Methods of Treatment Assignment

Patients in Phase 1 are assigned to the open dose cohort at the time of their enrollment. Patients in Phase 2 are assigned to Group 1 or Group 2 based upon whether they have a diagnosis of CRC (Group 1) or SCCHN (Group 2). Following assignment to Group 1 or Group 2, randomization will be made using a centralized system.

### Number of Planned Patients

Up to 162 evaluable patients are enrolled in this study. Phase 1 enrolls up to 18 patients in up to 3 cohorts during dose escalation. Up to 144 patients are enrolled in Phase 2 of the study, including 72 patients (36 patients per arm) in Group 1 (CRC) and 72 patients (36 patients per arm) in Group 2 (SCCHN).

### Duration of Treatment

Patients are evaluated for tumor response approximately every 6 weeks. Patients with stable disease (SD) or better per investigator assessment receive repeat cycles of treatment on a 21-day schedule until disease progression, unacceptable toxicity, or withdrawal of consent.

### Efficacy Assessments

Efficacy are evaluated per RECIST 1.1 (RECIST).

### Safety Assessments

Safety assessments include surveillance and documentation of adverse events, laboratory assessments, and physical exam findings.

### Pharmacokinetic Assessments

Pharmacokinetic (PK) assessments include measurement of serum levels of cetuximab in Phase 1. The effect of PX-866 administered at the MTD/RD on cetuximab peak and trough levels is evaluated in the Phase 2 population. Levels of PX-866 administered at the MTD/RD also are evaluated in Phase 2.

### Biomarker Assessments

Exploratory biomarker assessments will include evaluation of the tumor mutational profile, including but not limited to phosphatase and tensin homolog (PTEN) mutational status, PI3K gene amplification, PI3K catalytic subunit alpha (PIK3CA) mutational status, Kras mutational status, and Braf mutational status.

### Pharmacodynamic Assessments

Exploratory pharmacodynamic (PD) assessments in Phase 1 include evaluation of changes in activation of the PI3K and EGFR signaling pathways using PBMC, platelets, and optional paired tumor biopsies. Other PD assessments include changes in fasting C-peptide levels.

### Endpoints

### Phase 1

Primary Endpoint: Incidence and severity of adverse events

Secondary Endpoint:
Incidence and severity of clinical laboratory abnormalities
Disease control rate (DCR; the proportion of patients with CR, PR or SD) at 6, 12, 18 and 24 weeks
Objective response rate (ORR)
Early progression rate (% of patients with progressive disease at 6 weeks)
Duration of response
Serum concentrations of cetuximab
Frequency of dose reductions in cetuximab
Frequency of dose reductions in PX-866

### Phase 2

Primary Endpoint: Objective response rate (ORR)

Secondary Endpoint:
Progression free survival (PFS)
Incidence and severity of adverse events
Incidence and severity of clinical laboratory abnormalities
Duration of response
Disease control rate (DCR; the proportion of patients with CR, PR or SD) at 6, 12, 18 and 24 weeks
Overall survival
Early progression rate (% of patients with progressive disease at 6 weeks)
Frequency of dose reductions in cetuximab
Frequency of dose reductions in PX-866
Serum concentrations of cetuximab
Plasma concentrations of PX-866 and metabolites

### Phase 1 and Phase 2

Exploratory Endpoints
Tumor mutational profile, including but not limited to PTEN mutational status, PI3K gene amplification, PIK3CA mutational status, Kras mutational status, and Braf mutational status
Changes in phosphorylation status of EGFR and PI-3K pathway signaling proteins including but not limited to AKT, EGFR, mTOR, S6 in PBMCs, platelets and tumor biopsies (Phase 1 only)
Changes in fasting C-peptide levels

### Statistical Methods

Phase 1: Phase 1 of the study enrolls up to 18 patients in 3 cohorts during dose escalation (such that a minimum of 6 evaluable patients will have been treated at the MTD/RD). A dose is considered not tolerated if 2 or more patients experience a DLT.

Phase 2: Up to 144 patients are enrolled in Phase 2, including 72 patients in Group 1 with incurable metastatic CRC who have a history of progression or recurrence following prior irinotecan and oxaliplatin containing regimens or are intolerant of irinotecan randomized 1:1 to receive either PX-866+ cetuximab or cetuximab alone, and 72 patients in Group 2 with incurable progressive, recurrent or metastatic SCCHN randomized 1:1 to receive wither PX-866+ cetuximab or cetuximab alone. The sample size for Phase 2 is determined using a primary efficacy endpoint for either patient Group (i.e. tumor type or indication) of ORR. The hypothesis to be tested in each Group is H0: δ≦0 versus H1: δ>0. δ represents the population value of the difference in treatment proportions in the ORR. Thus, δ=P_{PX-866+C}−P_{c} where P is the population proportion of patients achieving an objective response. The minimal value of δ to detect within each Group is 0.20. P_{c} (the ORR in the cetuximab alone arm) will be set to 0.15 in deriving the sample size, effectively maximizing the calculation over the expected range of control arm responses. The study for each Group is designed as a screening trial with α (one-sided) and β error probabilities set to 0.20 each. Based on these considerations the required sample per Group is 72 patients; 36 patients will be randomized between the treatment arms within each Group.

### Evaluation of Safety

Adverse events are coded according to the Medical Dictionary for Regulatory Activities (MedDRA). Safety data are summarized by dose cohort and presented by individual treatment course as well as pooled over treatment courses. The severity of clinical adverse events and laboratory data are graded using NCI CTCAE, Version 4.02.

### Evaluation of Efficacy

Efficacy is evaluated according to RECIST. Independent radiological review can be performed on computed tomography (CT) or magnetic resonance imaging (MRI) scans from patients in Phase 2.

### Evaluation of Pharmacokinetic Activity

Non-compartmental pharmacokinetic modeling is used to estimate PK parameters for PX-866 and metabolites, as well as for cetuximab.

### Safety Monitoring Committee (SMC)

During the dose-escalation phase of the study, the SMC will meet to review adverse events and laboratory toxicities for the current dose cohort to determine if escalation to the next sequential dose cohort is warranted. The SMC also recommends the MTD/RD for Phase 2. During Phase 2 of the study, the SMC performs ongoing reviews of safety. The SMC meets every 12 weeks to review safety data and at additional times conditional on the accrual rate, results from previous meetings, or other items that may warrant additional meetings.

Efficacy data also is reviewed at the SMC meetings during the Phase 2 portion of the trial. These data is used to qualify the safety review by providing a global view of benefit and risk.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating a subject with a locally advanced, recurrent or metastatic cancer comprising administering to the subject a compound selected from 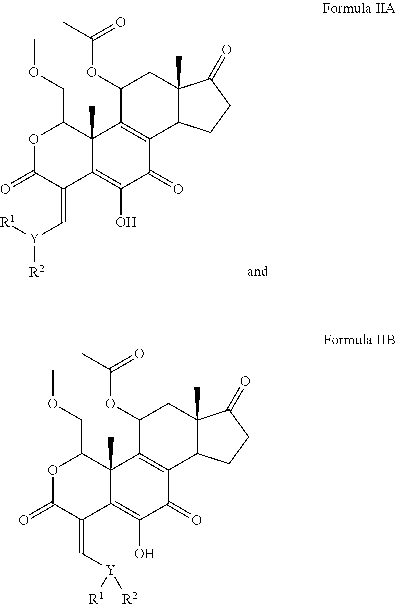
wherein Y is a heteroatom selected from nitrogen and sulfur and R¹ and R² are independently selected from an unsaturated alkyl, cyclic alkyl, or R¹ and R² together with Y form a heterocycle, and docetaxel.

2. The method of claim 1, wherein the cancer is selected from the group consisting of head and neck cancer, lung cancer, ovarian cancer, liver cancer, colon cancer, breast cancer, pancreatic cancer, kidney cancer, cervical cancer, uterine cancer, prostate cancer, esophageal cancer and gastric cancer.

3. The method of claim 1, wherein the cancer is non-small cell lung cancer or head and neck squamous cell carcinoma.

4. The method of claim 1 further comprising pretreating the subject with a corticosteroid prior to administration of the compound and docetaxel.

5. The method of claim 1 further comprising administering an additional anti-mitotic, platinum therapy or both.

6. The method of claim 1 further comprising an anti-emetic, anti-diarrheal or both.

7. The method of claim 1 further comprising preselecting the subject having completed first-line anti-cancer therapy.

8. The method of claim 1 further comprising evaluating the treated subject, wherein the evaluation comprises determining at least one of: (a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life.

9. The method of claim 1, wherein the compound is

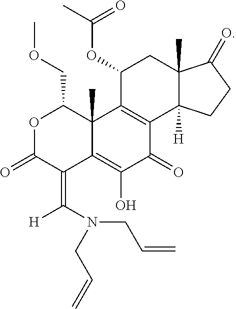 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

10. The method of claim 9, wherein the compound is administered at a dose and frequency sufficient to result in one or more of the following: 1) 17-hydroxy metabolite between about 500 pg/mL and about 2500 pg/mL (peak) within about 1-3 hours of administration; 2) plasma Cₘₐₓ of the 17-hydroxy metabolite of between about 750 pg/mL and about 1750 pg/mL; and 3) AUC of between about 2000 hr*pg/mL and about 8000 hr*pg/mL for the 17-hydroxy metabolite.

11. The method of any one of claim 1, wherein the compound is administered as a continuous dose, an intermittent dose or a combination thereof.

12. A method for treating a subject with a locally advanced, recurrent or metastatic cancer comprising administering to the subject a compound selected from 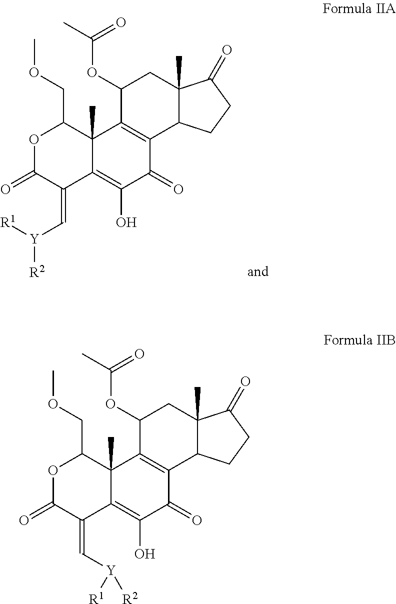
wherein Y is a heteroatom selected from nitrogen and sulfur and R¹ and R² are independently selected from an unsaturated alkyl, cyclic alkyl, or R¹ and R² together with Y form a heterocycle, and cetuximab.

13. The method of claim 12, wherein the cancer is selected from the group consisting of head and neck cancer, lung cancer, ovarian cancer, liver cancer, colon cancer, breast cancer, pancreatic cancer, kidney cancer, cervical cancer, uterine cancer, prostate cancer, esophageal cancer and gastric cancer.

14. The method of claim 12, wherein the cancer is colorectal cancer or head and neck squamous cell carcinoma.

15. The method of claim 12 further comprising pretreating the subject with a corticosteroid prior to administration of the compound and cetuxamib.

16. The method of claim 12 further comprising administering a topoisomerase inhibitor.

17. The method of claim 12 further comprising an anti-emetic, anti-diarrheal or both.

18. The method of claim 12 further comprising preselecting the subject having completed first-line anti-cancer therapy.

19. The method of claim 12 further comprising evaluating the treated subject, wherein the evaluation comprises determining at least one of: (a) tumor size, (b) tumor location, (c) nodal stage, (d) growth rate of the cancer, (e) survival rate of the subject, (f) changes in the subject's cancer symptoms, (g) changes in the subject's Prostate Specific Antigen (PSA) concentration, (h) changes in the subject's PSA concentration doubling rate, (i) changes in the subject's biomarkers, or (i) changes in the subject's quality of life.

20. The method of claim 12, wherein the compound is

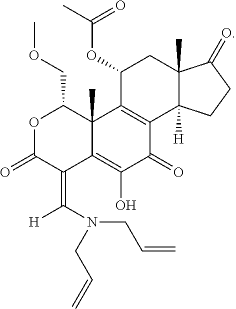 [SMILES: C=CCN(/C=C1/C(=O)O[C@H](COC)[C@@]2(C)C1=C(O)C(=O)C1=C2[C@H](OC(C)=O)C[C@]2(C)C(=O)CC[C@@H]12)CC=C]

21. The method of claim 20, wherein the compound is administered at a dose and frequency sufficient to result in one or more of the following: 1) 17-hydroxy metabolite between about 500 pg/mL and about 2500 pg/mL (peak) within about 1-3 hours of administration; 2) plasma Cₘₐₓ of the 17-hydroxy metabolite of between about 750 pg/mL and about 1750 pg/mL; and 3) AUC of between about 2000 hr*pg/mL and about 8000 hr*pg/mL for the 17-hydroxy metabolite.

22. The method of claim 1, wherein the compound is administered as a continuous dose, an intermittent dose or a combination thereof.
